(19) United States
(12) Patent Application Publication
(10) Pub. No.: US 2013/0195888 A1
(43) Pub. Date: Aug. 1, 2013

(54) ULTRAFILTRATION AND DIAFILTRATION FORMULATION METHODS FOR PROTEIN PROCESSING

(71) Applicants: Chen WANG, Shrewsbury, MA (US); Germano COPPOLA, Shrewsbury, MA (US); Johanna GERVAIS, West Townsend, MA (US); Robert K. HICKMAN, Worcester, MA (US); Roy D. HEGEDUS, Worcester, MA (US); Gregory J. BUNK, Holden, MA (US)

(72) Inventors: Chen WANG, Shrewsbury, MA (US); Germano COPPOLA, Shrewsbury, MA (US); Johanna GERVAIS, West Townsend, MA (US); Robert K. HICKMAN, Worcester, MA (US); Roy D. HEGEDUS, Worcester, MA (US); Gregory J. BUNK, Holden, MA (US)

(73) Assignee: AbbVie, North Chicago, IL (US)

(21) Appl. No.: 13/794,881

(22) Filed: Mar. 12, 2013

Related U.S. Application Data
(63) Continuation-in-part of application No. 12/325,049, filed on Nov. 28, 2008, now Pat. No. 8,420,081.
(60) Provisional application No. 61/004,992, filed on Nov. 30, 2007.

(51) Int. Cl. C07K 1/34

(52) U.S. Cl. CPC C07K 1/34
USPC 424/158.1; 530/414; 530/389.2; 530/389.1; 424/130.1

(57) ABSTRACT

Disclosed herein are methods of purifying proteins using ultrafiltration and diafiltration processes.

### RELATED APPLICATIONS

This application claims the benefit of priority to U.S. application Ser. No. 12/325,049 which claims priority to U.S. Provisional Application No. 61/004,992, filed on Nov. 30, 2007. The contents of the priority application are hereby incorporated by reference.

### BACKGROUND OF THE INVENTION

Often, protein-based pharmaceutical products need to be formulated at high concentrations for therapeutic efficacy. Highly concentrated protein formulations are desirable for therapeutic uses since they allow for dosages with smaller volumes, limiting patient discomfort, and are more economically packaged and stored. The development of high protein concentration formulations, however, presents many challenges, including manufacturing, stability, analytical, and, especially for therapeutic proteins, delivery challenges. For example, difficulties with the aggregation, insolubility and degradation of proteins generally increase as protein concentrations in formulations are raised (for review, see Shire, S. J. et al. J. Pharm. Sci., 93, 1390 (2004)). Previously unseen negative effects may be caused by additives that, at lower concentrations of the additives or the protein, provided beneficial effects. The production of high concentration protein formulations may lead to significant problems with opalescence, aggregation and precipitation during and/or after the processing. In addition to the potential for normative protein aggregation and particulate formation, reversible self-association may occur, which may result in increased viscosity or other properties that complicate delivery by injection. High viscosity also may complicate manufacturing of high protein concentrations by filtration approaches. High viscosity formulations may also have limited therapeutic administration.

Thus, pharmaceutical protein formulations typically carefully balance ingredients and concentrations to enhance protein stability and therapeutic requirements while limiting any negative side-effects. Biologic formulations should include stable protein, even at high concentrations, with specific amounts of excipients for reducing potential therapeutic complications, storage issues and overall cost.

Ultrafiltration and diafiltration (UF/DF) procedures are often used to produce pharmaceutical protein formulations at high concentrations. The formulation buffer salt and other excipients (e.g. cryoprotectant) required for achieving the desired pharmaceutical protein formulations are often introduced during the UF/DF process. One of the challenges associated with delivering pharmaceutical protein formulations at high concentrations is that, when protein is concentrated during the UF operation, significant volume exclusion and charge-charge interaction between the proteins and the charged buffer components can result in preferential distribution of charged buffer components into the permeate; the latter is the so-called “Donnan” effect. The Donnan effect causes a substantially lower level of charged buffer components in the product containing retentate than that in the diafiltration buffer. To compensate for this loss, a diafiltration buffer with a somewhat higher level of charged buffer components than the desired concentration in the final composition needs to be used in order to meet the desired formulation composition. Thus, the Donnan effect causes an increase in the ab initio calculated amount of buffer components that are used in protein-based pharmaceutical products.

Additionally, because such exclusion effect is dependent upon protein surface charge and concentration, each different protein-based pharmaceutical molecule may require different buffers for diafiltration. The determination of this diafiltration buffer concentration usually requires iterative experiments, necessitating a significant amount of materials and development effort. Thus, reconfiguration with new buffers and amounts of buffers is required every time a new protein purification process is scaled-up for larger scale manufacturing. Hence, there is a need to develop a method of UF/DF process that minimizes developmental efforts and requires fewer materials.

Another challenge that is often encountered during UF/DF processing is protein aggregation, precipitation and sub-visible particle formation, which can compromise product quality to the point of impacting safety and efficacy. Furthermore such product quality changes also complicate manufacturing through membrane fouling and result in product losses. Therefore, methods that can minimize or eliminate such product quality changes during the UF/DF formulation processing are highly desirable.

### SUMMARY OF THE INVENTION

This invention is directed towards the methods and processes for generating high concentration protein solutions with controlled formulation compositions. The methods and processes disclosed herein are based on ultrafiltration and diafiltration process, but can be applied to other concentration and buffer-exchange methods including centrifugation based concentration and dialysis. The invention also discloses methods for controlling protein aggregation and particle formation during the UF/DF processing. As exemplified below, this approach is effective for multiple monoclonal antibodies (mAbs) and dual-variable-domain immunoglobulins (DVD-Igs), and can be used to formulate protein solutions without affecting product quality and stability.

In an embodiment, the protein solution comprises one or more proteins. In an embodiment, UF/DF processes are disclosed wherein water is used as a diafiltration solvent for protein feed along with an optional step of adding (also referred to herein as “spiking”) an amount of a concentrated buffer solution into a concentrated retentate to achieve a final desired formulation. In an embodiment, at least a five-fold volume exchange with the diafiltration solvent is achieved. In an embodiment, the one or more proteins are concentrated to within a range of about 10 grams per liter to about 300 grams per liter. In an embodiment, the protein solution is concentrated by ultrafiltration.

In an embodiment, one or more buffer salts and/or one or more excipients are added during the UF/DF processing. In an embodiment, the one or more buffer salts comprises histidine within a concentration range of about 10 millimolar to about 100 millimolar. In an embodiment, histidine is added in a sufficient amount to achieve a target concentration of about 15 mM histidine at a target pH of about 6 in the formulation. In an embodiment, the protein formulation does not comprise a tonicity modifier, an anti-oxidant, a cryoprotectant, a bulking agent, or a lyoprotectant In an embodiment, the formulation is stable in a liquid form for at least about 3 months. In an embodiment, methods to control and mitigate protein aggregation and turbidity/particle formation that often occur during the UF/DF process are disclosed. One method is to reduce feed pH using acetic acid and/or citric acid (e.g. to adjust feed to pH range of about 4-6) prior to starting UF/DF processing. The pH-adjusted protein solution can be diafiltered either using water or a buffer as the diafiltration medium.

In another embodiment, methods of adding a low level of surfactant solution during the UF/DF operation are disclosed to mitigate protein aggregation and particle formation. Specifically, one method disclosed herein is to diafilter antibodies directly into a low level of a polysorbate aqueous solution, e.g. about 0.0001% to about 0.5%, (w/v) while another disclosed method is to add a low level of a polysorbate aqueous solution, e.g. about 0.0001% to about 0.5% (w/v), into the protein solution prior to starting the diafiltration processing.

In an embodiment, the one or more proteins is an antibody, or antigen binding fragment thereof. In an embodiment, the antibody, or antigen-binding fragment thereof, is selected from the group consisting of a chimeric antibody, a human antibody, a humanized antibody, and a domain antibody (dAb). In an embodiment, the antibody, or antigen-binding fragment thereof, is an anti-TNF or an anti-IL-12 antibody. In an embodiment, the antibody, or antigen-binding fragment thereof, is selected from the group consisting of Humira (adalimumab), Campath (Alemtuzumab), CEA-Scan Arcitumomab (fab fragment), Erbitux (Cetuximab), Herceptin (Trastuzumab), Myoscint (Imciromab Pentetate), ProstaScint (Capromab Pendetide), Remicade (Infliximab), ReoPro (Abciximab), Rituxan (Rituximab), Simulect (Basiliximab), Synagis (Palivizumab), Verluma (Nofetumomab), Xolair (Omalizumab), Zenapax (Daclizumab), Zevalin (Ibritumomab Tiuxetan), Orthoclone OKT3 (Muromonab-CD3), Panorex (Edrecolomab), Mylotarg (Gemtuzumab ozogamicin), golimumab (Centocor), Cimzia (Certolizumab pegol), Saris (Eculizumab), CNTO 1275 (ustekinumab), Vectibix (panitumumab), Bexxar (tositumomab and 1131 tositumomab), Avastin (bevacizumab), 13C5.5, CPA4026, PG110, 111-10, and DVD12-1CHO.

In an embodiment, the one or more proteins is a therapeutic protein selected from the group consisting of Pulmozyme (Dornase alfa), Rebif, Regranex (Becaplermin), Activase (Alteplase), Aldurazyme (Laronidase), Amevive (Alefacept), Aranesp (Darbepoetin alfa), Becaplermin Concentrate, Betaseron (Interferon beta-1b), BOTOX (Botulinum Toxin Type A), Elitek (Rasburicase), Elspar (Asparaginase), Epogen (Epoetin alfa), Enbrel (Etanercept), Fabrazyme (Agalsidase beta), Infergen (Interferon alfacon-1), Intron A (Interferon alfa-2a), Kineret (Anakinra), MYOBLOC (Botulinum Toxin Type B), Neulasta (Pegfilgrastim), Neumega (Oprelvekin), Neupogen (Filgrastim), Ontak (Denileukin diftitox), PEGASYS (Peginterferon alfa-2a), Proleukin (Aldesleukin), Pulmozyme (Dornase alfa), Rebif (Interferon beta-1a), Regranex (Becaplermin), Retavase (Reteplase), Roferon-A (Interferon alfa-2), TNKase (Tenecteplase), and Xigris (Drotrecogin alfa), Arcalyst (Rilonacept), NPlate (Romiplostim), Mircera (methoxypolyethylene glycol-epoetin beta), Cinryze (C1 esterase inhibitor), Elaprase (idursulfase), Myozyme (alglucosidase alfa), Orencia (abatacept), Naglazyme (galsulfase), Kepivance (palifermin), and Actimmune (interferon gamma-1b).

In an embodiment, the protein formulation may be administered to a subject in an effective amount to treat a disease or disorder. In an embodiment, the disease or disorder is rheumatoid arthritis, juvenile idiopathic arthritis, psoriatic arthritis, osteoarthritis, Crohn's disease, ulcerative colitis, ankylosing spondylitis, psoriasis, multiple sclerosis, sarcoidosis, neutropenia, leukemia, lymphoma, a central neurological disorder, a peripheral neurological disorder, chronic pain, acute pain, lung cancer, stomach cancer, colon cancer, prostate cancer, brain cancer, or breast cancer. In another embodiment, the protein formulation comprises a device.

The methods disclosed herein can be incorporated individually as separate steps or in multiple combinations into various UF/DF approaches to further improve processing yield/throughput and product quality of antibody or other protein-based pharmaceutical products.

### DETAILED DESCRIPTION OF THE INVENTION

The present disclosure relates to methods and compositions for generating pharmaceutical protein formulations at high concentrations. Specifically, the methods and compositions of the present disclosure are based on a diafiltration process wherein a first solution containing the protein of interest is diafiltered using water as a diafiltration medium. The process is performed such that there is at least a determined volume exchange, e.g., a five-fold volume exchange, with the water. By performing the methods of the present disclosure, the resulting aqueous formulation has a significant decrease in the overall percentage of excipients in comparison to the initial protein solution. For example, 95-99% less excipients are found in the aqueous formulation in comparison to the initial protein solution. Despite the decrease in excipients, the protein remains soluble and retains its biological activity, even at high concentrations. In one aspect, the methods of the present disclosure result in compositions comprising an increase in concentration of the protein while decreasing additional components, such as ionic excipients. As such, the hydrodynamic diameter of the protein in the aqueous formulation is smaller relative to the same protein in a standard buffering solution, such as phosphate buffered saline (PBS).

The formulation of the present disclosure has many advantages over standard buffered formulations. In one aspect, the aqueous formulation comprises high protein concentrations, e.g., 50 to 200 mg/mL or more. Proteins of all sizes may be included in the formulations of the present disclosure, even at increased concentrations. Despite the high concentration of protein, the formulation has minimal aggregation and can be stored using various methods and forms, e.g., freezing, without deleterious effects that might be expected with high protein formulations. In one embodiment, the formulations of the present disclosure do not require excipients, such as, for example, surfactants and buffering systems, which are used in traditional formulations to stabilize proteins in solution. As a result of the low level of ionic excipients, the aqueous formulation of the present disclosure has low conductivity, e.g., less than 2 mS/cm. The methods and compositions of the present disclosure also provide aqueous protein formulations having low osmolality, e.g., no greater than 30 mOsmol/kg. In addition, the formulations described herein are useful because they have decreased immunogenicity over standard formulations due to the lack of additional agents needed for protein stabilization.

The methods and compositions of the present disclosure may be used to provide an aqueous formulation comprising water and any type of protein of interest. In one aspect, the methods and compositions of the present disclosure are used for large proteins, including proteins which are larger than 47 kDa. Antibodies, and fragments thereof, including those used for in vivo and in vitro purposes, are another example of proteins which may be used in the methods and compositions of the present disclosure.

Furthermore, the multiple step purification and concentration processes that are necessary to prepare proteins and peptide formulations often introduce variability in compositions, such that the precise composition of a formulation may vary from lot to lot. Federal regulations require that drug compositions be highly consistent in their formulations regardless of the location of manufacture or lot number. Methods of the present disclosure can be used to create solutions of proteins formulated in water to which buffers and excipients are added back in precise amounts, allowing for the creation of protein formulations with precise concentrations of buffers and/or excipients.

Any protein may be used in the methods and compositions of the present disclosure. In one embodiment, the formulation comprises a therapeutic protein. In one embodiment, the formulation comprises an antibody, or an antigen-binding fragment thereof. Types of antibodies, or antigen binding fragments, that may be included in the methods and compositions of the present disclosure include, but are not limited to, a chimeric antibody, a human antibody, a humanized antibody, and a domain antibody (dAb). In one embodiment, the antibody, DVD-Ig, or antigen-binding fragment thereof, is an anti-TNFα antibody, such as but not limited to adalimumab, certolizumab pegol, etanercept, infliximab, golimumab, or abatacept, or an anti-IL-12 antibody, such as but not limited to J695 or ustekinumab, or other antibodies such as 13C5.5, CPA4026, PG110, or 111-10, or DVD12-1CHO, for example. In addition, the formulation of the present disclosure may also include at least two distinct types of proteins, e.g., adalimumab and J695.

The formulation of the present disclosure may be suitable for any use, including both in vitro and in vivo uses. In one embodiment, the formulation of the present disclosure is suitable for administration to a subject via a mode of administration, including, but not limited to, subcutaneous, intravenous, inhalation, intradermal, transdermal, intraperitoneal, and intramuscular administration. The formulation of the present disclosure may be used in the treatment of a disorder in a subject.

Also included in the present disclosure are devices that may be used to deliver the formulation of the present disclosure. Examples of such devices include, but are not limited to, a syringe, a pen, an implant, a needle-free injection device, an inhalation device, and a patch. In one embodiment, the formulation of the present disclosure is a pharmaceutical formulation.

The present disclosure further provides a method of preparing an aqueous formulation of a protein, such as an antibody. In an embodiment the method comprises providing the protein in a first solution; subjecting the first solution to diafiltration using water as a diafiltration medium until at least a five-fold volume exchange with the water has been achieved to thereby prepare a diafiltered protein solution; and concentrating the diafiltered protein solution using ultrafiltration to thereby prepare the aqueous formulation of the protein. In an embodiment, the protein in the resulting formulation retains its biological activity.

In one embodiment, the first solution is subjected to diafiltration with water until a volume exchange greater than a five-fold volume exchange is achieved. In one embodiment, the first solution is subjected to diafiltration with water until at least about a six-fold volume exchange is achieved. In one embodiment, the first solution is subjected to diafiltration with water until at least about an eight-fold volume exchange is achieved.

In an embodiment, the aqueous formulation has a final concentration of excipients which is at least about 95% less than the first solution. In another embodiment, the aqueous formulation has a final concentration of excipients which is at least about 99% less than the first solution.

In an embodiment, the first solution is obtained from a mammalian cell expression system and has been purified to remove host cell proteins (HCPs). In another embodiment, the first solution may be further concentrated prior to the UF/DF process.

In yet another embodiment, the method of the present disclosure further comprises adding an excipient to the aqueous formulation. In an embodiment, the method of the present disclosure involves adding buffer salts to the aqueous formulation. In an embodiment, the method of the present disclosure further comprises adding histidine by spiking the concentrated solution at some point during the UF/DF process. In an embodiment, the diafiltration buffer comprises histidine in a concentration from about 10 millimolar to about 100 millimolar.

In an embodiment, an acid is added to adjust the pH of the protein solutions prior to initiating the UF/DF formulation process according to the methods disclosed herein. In an embodiment, citric acid and/or acetic acid are used to decrease the turbidity of a protein solution that is developed during the UF/DF process disclosed herein. In an embodiment, acetic acid and/or citric acid are used to adjust the pH of an antibody or other protein containing solution to a pH of from about 4 to about 8. In an embodiment, acetic acid and/or citric acid are used to adjust the pH of an antibody or other protein containing solution to a pH of from about 5 to about 6.

In another embodiment, a surfactant is used to decrease the turbidity of a protein solution that is developed during the UF/DF processes disclosed herein. In an embodiment, polysorbate is used at concentration from about 0.0001% to about 0.5% (w/v) at various steps of the UF/DF processes disclosed herein.

Many mAbs and DVD-Igs produced require a formulation of high protein concentration (≧70 g/L) with defined buffer compositions. In an embodiment, a drug substance (DS) formulation consists of 15 mM histidine at target pH of about 6. This is partially achieved through UF/DF operations and steps that use an ultrafiltration membrane with proper molecular weight cut off (MWCO). In an embodiment, the protein feed is first concentrated (e.g. to about 50 g/L), then diafiltered into a histidine buffer followed by a second ultrafiltration step to achieve a final targeted protein concentration.

In an embodiment, water is used as a diafiltration solvent for antibody feed along with adding (also referred to herein as “spiking”) a proper amount of a concentrated histidine solution into the concentrated retentate to achieve a final DS formulation. As exemplified below, this approach is effective for multiple mAbs and DVD-Igs, and can be used to formulate a drug substance or ready-to-fill drug product without affecting product quality and stability.

In an embodiment, methods to control and mitigate antibody aggregation and turbidity formation that often occur during the UF/DF process are disclosed. One method is to reduce feed pH using acetic acid and/or citric acid (e.g. to adjust feed to pH range of about 5-6) prior to starting UF/DF processing. Another method is to diafilter antibodies directly into a low level of a polysorbate aqueous solution, e.g. from about 0.0001% to about 0.5% (w/v). Yet another method is to add a low level of a polysorbate aqueous solution, e.g., from about 0.0001% to about 0.5% (w/v), into protein solution prior to starting the diafiltration operation. These methods can be incorporated into a UF/DF approach to further improve processing yield/throughput and product quality. Additionally, embodiments disclosed herein present methods of UF/DF processing that minimize developmental efforts as well as decreasing material requirements for the purification of various proteins, e.g., mAbs and DVD-Igs.

### DEFINITIONS

In order that the present disclosure may be more readily understood, certain terms are first defined.

The term “aqueous formulation” refers to a solution in which the solvent is water.

As used herein, the term “acidic component” refers to an agent, including a solution, having an acidic pH, i.e., less than 7.0. Examples of acidic components include phosphoric acid, hydrochloric acid, acetic acid, citric acid, oxalic acid, succinic acid, tartaric acid, lactic acid, malic acid, glycolic acid and fumaric acid. In one embodiment, the aqueous formulation of the present disclosure does not include an acidic component.

As used herein, the term “basic component” refers to an agent which is alkaline, i.e., pH greater than 7.0. Examples of basic components include potassium hydroxide (KOH) and sodium hydroxide (NaOH).

The term “conductivity,” as used herein, refers to the ability of an aqueous solution to conduct an electric current between two electrodes. Generally, electrical conductivity or specific conductivity is a measure of a material's ability to conduct an electric current. In solution, the current flows by ion transport. Therefore, with an increasing amount of ions present in the aqueous solution, the solution will have a higher conductivity. The unit of measurement for conductivity is mmhos (mS/cm), and can be measured using a conductivity meter sold, e.g., by Orion Research, Inc. (Beverly, Mass.). The conductivity of a solution may be altered by changing the concentration of ions therein. For example, the concentration of ionic excipients in the solution may be altered in order to achieve the desired conductivity.

The term “cryoprotectants” as used herein generally includes agents, which provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations.

As used herein, the terms “ultrafiltration” or “UF” refers to any technique in which a solution or a suspension is subjected to a semi-permeable membrane that retains macromolecules while allowing solvent and small solute molecules to pass through. Ultrafiltration may be used to increase the concentration of macromolecules in a solution or suspension. In an aspect, ultrafiltration is used to increase the concentration of a protein in water.

As used herein, the term “diafiltration” or “DF” is used to mean a specialized class of filtration in which the retentate is diluted with solvent and re-filtered, to reduce the concentration of soluble permeate components. Diafiltration may or may not lead to an increase in the concentration of retained components, including, proteins. For example, in continuous diafiltration, a solvent is continuously added to the retentate at the same rate as the filtrate is generated. In this case, the retentate volume and the concentration of retained components does not change during the process. On the other hand, in discontinuous or sequential dilution diafiltration, an ultrafiltration step is followed by the addition of solvent to the retentate side; if the volume of solvent added to the retentate side is not equal or greater to the volume of filtrate generated, then the retained components will have a high concentration. Diafiltration may be used to alter the pH, ionic strength, salt composition, buffer composition, or other properties of a solution or suspension of macromolecules.

As used herein, the terms “diafiltration/ultrafiltration” or “DF/UF” refer to any process, technique or combination of techniques that accomplishes ultrafiltration and/or diafiltration, either sequentially or simultaneously.

As used herein, the term “diafiltration volume” refers to a total volume exchange during the process of diafiltration.

The term “excipient” refers to an agent that may be added to a formulation to provide a desired consistency, (e.g., altering the bulk properties), to improve stability, and/or to adjust osmolality. Examples of commonly used excipients include, but are not limited to, sugars, polyols, amino acids, surfactants, and polymers. The term “ionic excipient” or “ionizable excipient,” as used interchangeably herein, refers to an agent that has a net charge. In one embodiment, the ionic excipient has a net charge under certain formulation conditions, such as pH. Examples of an ionic excipient include, but are not limited to, histidine, arginine, glycine, sodium phosphate and sodium chloride. The term “non-ionic excipient” or “non-ionizable excipient,” as used interchangeably herein, refers to an agent having no net charge. In one embodiment, the non-ionic excipient has no net charge under certain formulation conditions, such as pH. Examples of non-ionic excipients include, but are not limited to, sugars (e.g., sucrose), sugar alcohols (e.g., mannitol), and non-ionic surfactants (e.g., polysorbate 80).

The term “first protein solution” or “first solution” as used herein, refers to the initial protein solution or starting material used in the methods of the present disclosure, e.g., the initial protein solution which is diafiltered into an aqueous solution. The first protein solution may be concentrated to an intermediate concentration before starting the diafiltration step. In one embodiment, the first protein solution comprises ionic excipients, non-ionic excipients, and/or a buffering system.

The term “hydrodynamic diameter” or “Dₕ” of a particle refers to the diameter of a sphere that has the density of water and the same velocity as the particle. Thus the term “hydrodynamic diameter of a protein” as used herein refers to a size determination for proteins in solution using dynamic light scattering (DLS). A DLS-measuring instrument measures the time-dependent fluctuation in the intensity of light scattered from the proteins in solution at a fixed scattering angle. Protein Dₕ is determined from the intensity autocorrelation function of the time-dependent fluctuation in intensity. Scattering intensity data are processed using DLS instrument software to determine the value for the hydrodynamic diameter and the size distribution of the scattering molecules, i.e. the protein specimen.

The term “lyoprotectant” as used herein includes agents that provide stability to a protein during water removal during the drying or lyophilisation process, for example, by maintaining the proper conformation of the protein. Examples of lyoprotectants include saccharides, in particular di- or trisaccharides. Cryoprotectants may also provide lyoprotectant effects.

The term “pharmaceutical” as used herein with reference to a composition, e.g., an aqueous formulation, that it is useful for treating a disease or disorder.

The term “protein” is meant to include a sequence of amino acids for which the chain length is sufficient to produce the higher levels of secondary and/or tertiary and/or quaternary structure. This is to distinguish from “peptides” or other small molecular weight drugs that do not have such structure. In one embodiment, the proteins used herein have a molecular weight of at least about 47 kD. Examples of proteins encompassed within the definition used herein include therapeutic proteins. A “therapeutically active protein” or “therapeutic protein” refers to a protein which may be used for therapeutic purposes, i.e., for the treatment of a disorder in a subject. It should be noted that while therapeutic proteins may be used for treatment purposes, the present disclosure is not limited to such use, as said proteins may also be used for in vitro studies. In an aspect, the therapeutic protein is a fusion protein or an antibody, or antigen-binding portion thereof. In one embodiment, the methods and compositions of the present disclosure comprise at least two distinct proteins, which are defined as two proteins having distinct amino acid sequences. Additional distinct proteins do not include degradation products of a protein.

The phrase “protein is dissolved in water” as used herein refers to a formulation of a protein wherein the protein is dissolved in an aqueous solution in which the amount of small molecules (e.g., buffers, excipients, salts, surfactants) has been reduced by DF/UF processing. Even though the total elimination of small molecules cannot be achieved in an absolute sense by DF/UF processing, the theoretical reduction of excipients achievable by applying DF/UF is sufficiently large to create a formulation of the protein essentially in water exclusively. For example, with 6 volume exchanges in a continuous mode DF/UF protocol, the theoretical reduction of excipients is about 99.8% (C_{f}=Cᵢ·e^{-x}, with C_{f} and Cᵢ being the final and initial excipient concentrations, respectively, and x being the number of volume exchanges).

The term “pharmaceutical formulation” refers to preparations which are in such a form as to permit the biological activity of the active ingredients to be effective, and, therefore may be administered to a subject for therapeutic use.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. In one embodiment, the stability of the protein is determined according to the percentage of monomer protein in the solution, with a low percentage of degraded (e.g., fragmented) and/or aggregated protein. For example, an aqueous formulation comprising a stable protein may include at least 95% monomer protein. Alternatively, an aqueous formulation of the present disclosure may include no more than 5% aggregate and/or degraded protein.

The term “stabilizing agent” refers to an excipient that improves or otherwise enhances stability. Stabilizing agents include, but are not limited to, α-lipoic acid, α-tocopherol, ascorbyl palmitate, benzyl alcohol, biotin, bisulfites, boron, butylated hydroxyanisole (BHA), butylated hydroxytoluene (BHT), ascorbic acid and its esters, carotenoids, calcium citrate, acetyl-L-camitine, chelating agents, chondroitin, chromium, citric acid, coenzyme Q-10, cysteine, cysteine hydrochloride, 3-dehydroshikimic acid (DHS), EDTA (ethylenediaminetetraacetic acid; edetate disodium), ferrous sulfate, folic acid, fumaric acid, alkyl gallates, garlic, glucosamine, grape seed extract, gugul, magnesium, malic acid, metabisulfite, N-acetyl cysteine, niacin, nicotinomide, nettle root, ornithine, propyl gallate, pycnogenol, saw palmetto, selenium, sodium bisulfite, sodium metabisulfite, sodium sulfite, potassium sulfite, tartaric acid, thiosulfates, thioglycerol, thiosorbitol, tocopherol and their esters, e.g., tocopheral acetate, tocopherol succinate, tocotrienal, d-α-tocopherol acetate, vitamin A and its esters, vitamin B and its esters, vitamin C and its esters, vitamin D and its esters, vitamin E and its esters, e.g., vitamin E acetate, zinc, and combinations thereof.

The term “surfactants” generally includes those agents that protect the protein from air/solution interface-induced stresses and solution/surface induced-stresses. For example surfactants may protect the protein from aggregation. Suitable surfactants may include, e.g., polysorbates, polyoxyethylene alkyl ethers such as Brij 35™, or poloxamer such as Tween 20, Tween 80, or poloxamer 188. Detergents include, but are not limited to, poloxamers, e.g., Poloxamer 188, Poloxamer 407; polyoxyethylene alkyl ethers, e.g., Brij 35™, Cremophor A25, Sympatens ALM/230; and polysorbates/Tweens, e.g., Polysorbate 20, Polysorbate 80, and Poloxamers, e.g., Poloxamer 188, and Tweens, e.g., Tween 20 and Tween 80.

As used herein, the term “tonicity modifier” is intended to mean a compound or compounds that can be used to adjust the tonicity of a liquid formulation. Suitable tonicity modifiers include glycerin, lactose, mannitol, dextrose, sodium chloride, magnesium sulfate, magnesium chloride, sodium sulfate, sorbitol, trehalose, sucrose, raffinose, maltose and others known to those or ordinary skill in the art. In one embodiment, the tonicity of the liquid formulation approximates that of the tonicity of blood or plasma.

The term “water” is intended to mean water that has been purified to remove contaminants, usually by distillation or reverse osmosis, also referred to herein as “pure water”. In an aspect, water used in the methods and compositions of the present disclosure is excipient-free. In one embodiment, water includes sterile water suitable for administration to a subject. In another embodiment, water is meant to include water for injection (WFI). In one embodiment, water refers to distilled water or water which is appropriate for use in in vitro assays. In an aspect, diafiltration is performed in accordance with the methods of the present disclosure using water alone as the diafiltration medium.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., TNFα, IL-12). It has been shown that the antigen-binding function of an antibody can be performed by fragments of a full-length antibody. Examples of binding fragments encompassed within the term “antigen-binding portion” of an antibody include: (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al, (1989) Nature 341:544-546), which consists of a V_{H} or V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. In one embodiment of the present disclosure, the antibody fragment is selected from the group consisting of an Fab, an Fd, an Fd′, a single chain Fv (scFv), an scFva, and a domain antibody (dAb).

Still further, an antibody or antigen-binding portion thereof may be part of a larger immunoadhesion molecule, formed by covalent or noncovalent association of the antibody or antibody portion with one or more other proteins or peptides. These other proteins or peptides can have functionalities that allow for the purification of antibodies or antigen-binding portions thereof or allow for their association with each other or other molecules. Thus, examples of such immunoadhesion molecules include use of the streptavidin core region to make a tetrameric single chain variable fragment (scFv) molecules (Kipriyanov et al. (1995) Human Antibodies and Hybridomas 6:93-101) and the use of a cysteine residue, a marker peptide and a C-terminal polyhistidine tag to make bivalent and biotinylated scFv molecules (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Two antibody domains are “complementary” where they belong to families of structures which form cognate pairs or groups or are derived from such families and retain this feature. For example, a V_{H} domain and a V_{L} domain of an antibody are complementary; two V_{H} domains are not complementary, and two V_{L} domains are not complementary. Complementary domains may be found in other members of the immunoglobulin superfamily, such as the V_{H} and Vβ (or gamma and delta) domains of the T-cell receptor.

The term “domain” refers to a folded protein structure which retains its tertiary structure independently of the rest of the protein. Generally, domains are responsible for discrete functional properties of proteins, and in many cases may be added, removed or transferred to other proteins without loss of function of the remainder of the protein and/or of the domain. By single antibody variable domain is meant a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least in part the binding activity and specificity of the full-length domain.

Variable domains of the present disclosure may be combined to form a group of domains; for example, complementary domains may be combined, such as V_{L} domains being combined with V_{H} domains. Non-complementary domains may also be combined. Domains may be combined in a number of ways, involving linkage of the domains by covalent or non-covalent means.

A “dAb” or “domain antibody” refers to a single antibody variable domain (V_{H} or V_{L}) polypeptide that specifically binds antigen.

As used herein, the term “antigen binding region” or “antigen binding site” refers to the portion(s) of an antibody molecule, or antigen binding portion thereof, which contains the amino acid residues that interact with an antigen and confers on the antibody its specificity and affinity for the antigen.

The term “epitope” is meant to refer to that portion of any molecule capable of being recognized by and bound by an antibody at one or more of the antibody's antigen binding regions. In the context of the present disclosure, first and second “epitopes” are understood to be epitopes which are not the same and are not bound by a single monospecific antibody, or antigen-binding portion thereof.

The term “DVD-Ig” or “dual-variable-domain-immunoglobulin” refers to a protein containing two or more antigen binding sites and is a tetravalent or multivalent binding protein. The DVD-Ig comprises two heavy chain DVD polypeptides and two light chain DVD polypeptides; each half of a DVD-Ig comprises a heavy chain DVD polypeptide, and a light chain DVD polypeptide, and two antigen binding sites. Each binding site comprises a heavy chain variable domain and a light chain variable domain with a total of six CDRs involved in antigen binding per antigen binding site. The description and generation of DVD-Ig molecules are detailed in the U.S. Pat. No. 7,612,181 B2.

The phrase “recombinant antibody” refers to antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of particular immunoglobulin gene sequences (such as human immunoglobulin gene sequences) to other DNA sequences. Examples of recombinant antibodies include chimeric, CDR-grafted and humanized antibodies.

The term “human antibody” refers to antibodies having variable and constant regions corresponding to, or derived from, human germline immunoglobulin sequences as described by, for example, Kabat et al. (See Kabat, et al. (1991) Sequences of Proteins of Immunological Interest, Fifth Edition, U.S. Department of Health and Human Services, NIH Publication No. 91-3242). The human antibodies of the present disclosure, however, may include amino acid residues not encoded by human germline immunoglobulin sequences (e.g., mutations introduced by random or site-specific mutagenesis in vitro or by somatic mutation in vivo), for example in the CDRs and in particular CDR3.

Recombinant human antibodies of the present disclosure have variable regions, and may also include constant regions, derived from human germline immunoglobulin sequences (See Kabat et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis or backmutation or both.

The term “backmutation” refers to a process in which some or all of the somatically mutated amino acids of a human antibody are replaced with the corresponding germline residues from a homologous germline antibody sequence. The heavy and light chain sequences of a human antibody of the present disclosure are aligned separately with the germline sequences in the VBASE database to identify the sequences with the highest homology. Differences in the human antibody of the present disclosure are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the human antibody should not be included in the final human antibody. To minimize the number of amino acids subject to backmutation those amino acid positions found to be different from the closest germline sequence but identical to the corresponding amino acid in a second germline sequence can remain, provided that the second germline sequence is identical and colinear to the sequence of the human antibody of the present disclosure for at least 10, up to about 12 amino acids, on both sides of the amino acid in question. Backmuation may occur at any stage of antibody optimization.

The term “chimeric antibody” refers to antibodies which comprise heavy and light chain variable region sequences from one species and constant region sequences from another species, such as antibodies having murine heavy and light chain variable regions linked to human constant regions.

The term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences.

Various aspects of the present disclosure are described below.

Generally, diafiltration is a technique that uses membranes to remove, replace, or lower the concentration of salts or solvents from solutions containing proteins, peptides, nucleic acids, and other biomolecules. Protein production operations often involve final diafiltration of a protein solution into a formulation buffer once the protein has been purified from impurities resulting from its expression, e.g., host cell proteins. The present disclosure described herein provides a means for obtaining an aqueous formulation by subjecting a protein solution to diafiltration using water alone as a diafiltration solution. Thus, the formulation of the present disclosure is based on using water as a formulation medium during the diafiltration process and does not rely on traditional formulation mediums which include excipients, such as buffer salt or cryoprotectant, used to solubilize and/or stabilize the protein in the final formulation. The present disclosure provides a method for transferring a protein into pure water for use in a stable formulation, wherein the protein remains in solution and is able to be concentrated at high levels without the use of other agents to maintain its stability.

Prior to diafiltration or DF/UF in accordance with the teachings herein, the method includes first providing a protein in a first solution. The protein may be formulated in any first solution, including formulations using techniques that are well established in the art, such as synthetic techniques (e.g., recombinant techniques, peptide synthesis, or a combination thereof). Alternatively, the protein used in the methods and compositions of the present disclosure is isolated from an endogenous source of the protein. The initial protein solution may be obtained using a purification process whereby the protein is purified from a heterogeneous mixture of proteins. In one embodiment, the initial protein solution used in the present disclosure is obtained from a purification method whereby proteins, including antibodies, expressed in a mammalian expression system are subjected to numerous chromatography steps which remove host cell proteins (HCPs) from the protein solution. In one embodiment, the first protein solution is obtained from a mammalian cell expression system and has been purified to remove host cell proteins (HCPs). Examples of methods of purification are described in U.S. application Ser. No. 11/732,918 (US 20070292442), incorporated by reference herein.

Proteins which may be used in the compositions and methods of the present disclosure may be any size, i.e., molecular weight (M_{w}). For example, the protein may have a M_{w} equal to or greater than about 1 kDa, a M_{w} equal to or greater than about 10 kDa, a M_{w} equal to or greater than about 47 kDa, a M_{w} equal to or greater than about 57 kDa, a M_{w} equal to or greater than about 100 kDa, a M_{w} equal to or greater than about 150 kDa, a M_{w} equal to or greater than about 200 kDa, or a M_{w} equal to or greater than about 250 kDa. Numbers intermediate to the above recited M_{w}, e.g., 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 153, 153, 154, 155, 156, 157, 158, 159, 160, 161, 162, 163, 164, 165, 166, 167, 168, 169, 170, and so forth, as well as all other numbers recited herein, are also intended to be part of this present disclosure. Ranges of values using a combination of any of the above recited values as upper and/or lower limits are intended to be included in the scope of the present disclosure. For example, proteins used in the present disclosure may range in size from 57 kDa to 250 kDa, from 56 kDa to 242 kDa, from 60 kDa to 270 kDa, and so forth.

The methods of the present disclosure also include diafiltration of a first protein solution that comprises at least two distinct proteins. For example, the protein solution may contain two or more types of antibodies directed to different molecules or different epitopes of the same molecule.

In one embodiment, the protein that is in solution is a therapeutic protein, including, but not limited to, fusion proteins and enzymes. Examples of therapeutic proteins include, but are not limited to, Pulmozyme (Dornase alfa), Regranex (Becaplermin), Activase (Alteplase), Aldurazyme (Laronidase), Amevive (Alefacept), Aranesp (Darbepoetin alfa), Becaplermin Concentrate, Betaseron (Interferon beta-1b), BOTOX (Botulinum Toxin Type A), Elitek (Rasburicase), Elspar (Asparaginase), Epogen (Epoetin alfa), Enbrel (Etanercept), Fabrazyme (Agalsidase beta), Infergen (Interferon alfacon-1), Intron A (Interferon alfa-2a), Kineret (Anakinra), MYOBLOC (Botulinum Toxin Type B), Neulasta (Pegfilgrastim), Neumega (Oprelvekin), Neupogen (Filgrastim), Ontak (Denileukin diftitox), PEGASYS (Peginterferon alfa-2a), Proleukin (Aldesleukin), Pulmozyme (Dornase alfa), Rebif (Interferon beta-1a), Regranex (Becaplermin), Retavase (Reteplase), Roferon-A (Interferon alfa-2), TNKase (Tenecteplase), and Xigris (Drotrecogin alfa), Arcalyst (Rilonacept), NPlate (Romiplostim), Mircera (methoxypolyethylene glycol-epoetin beta), Cinryze (C1 esterase inhibitor), Elaprase (idursulfase), Myozyme (alglucosidase alfa), Orencia (abatacept), Naglazyme (galsulfase), Kepivance (palifermin), and Actimmune (interferon gamma-1b).

The protein used in the present disclosure may also be an antibody, or antigen-binding fragment thereof. Examples of antibodies that may be used in the present disclosure include chimeric antibodies, non-human antibodies, human antibodies, humanized antibodies, and domain antibodies (dAbs). In one embodiment, the antibody, or antigen-binding fragment thereof, is an anti-TNFα and/or an anti-IL-12 antibody (e.g., it may be a dual variable domain (DVD) antibody). Other examples of antibodies and DVD-Igs, or antigen-binding fragments thereof, which may be used in the methods and compositions of the present disclosure include, but are not limited to, ID4.7 (anti-IL-12/IL-23 antibody; AbbVie), 2.5 (E)mg1 (anti-IL-18 antibody; AbbVie), 13C5.5 (anti-IL-13 antibody; AbbVie), J695 (anti-IL-12 antibody; AbbVie), Afelimomab (Fab 2 anti-TNF; AbbVie), Humira (adalimumab AbbVie), CPA4026 (anti-RGMa) antibody; AbbVie), PG110 (anti-NGF antibody; AbbVie), 111-10 (anti-EGFR antibody; AbbVie), DVD12-1CHO (anti-IL-1α/anti-IL-1β DVD-Ig; AbbVie), Campath (Alemtuzumab), CEA-Scan Arcitumomab (fab fragment), Erbitux (Cetuximab), Herceptin (Trastuzumab), Myoscint (Imciromab Pentetate), ProstaScint (Capromab Pendetide), Remicade (Infliximab), ReoPro (Abciximab), Rituxan (Rituximab), Simulect (Basiliximab), Synagis (Palivizumab), Verluma (Nofetumomab), Xolair (Omalizumab), Zenapax (Daclizumab), Zevalin (Ibritumomab Tiuxetan), Orthoclone OKT3 (Muromonab-CD3), Panorex (Edrecolomab), Mylotarg (Gemtuzumab ozogamicin), golimumab (Centocor), Cimzia (Certolizumab pegol), Soliris (Eculizumab), CNTO 1275 (ustekinumab), Vectibix (panitumumab), Bexxar (tositumomab and 1131 tositumomab), an anti-IL-17 antibody Antibody 7 as described in International Application WO 2007/149032 (Cambridge Antibody Technology), the entire contents of which are incorporated by reference herein, the anti-IL-13 antibody CAT-354 (Cambridge Antibody Technology), the anti-human CD4 antibody CE9y4PE (IDEC-151, clenoliximab) (Biogen IDEC/Glaxo Smith Kline), the anti-human CD4 antibody IDEC CE9.1/SB-210396 (keliximab) (Biogen IDEC), the anti-human CD80 antibody IDEC-114 (galiximab) (Biogen IDEC), the anti-Rabies Virus Protein antibody CR4098 (foravirumab), and the anti-human TNF-related apoptosis-inducing ligand receptor 2 (TRAIL-2) antibody HGS-ETR2 (lexatumumab) (Human Genome Sciences, Inc.), and Avastin (bevacizumab).

### Polyclonal Antibodies

Polyclonal antibodies generally refer to a mixture of antibodies that are specific to a certain antigen, but bind to different epitopes on said antigen. Polyclonal antibodies are generally raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R₁NCNR, where R and R₁ are different alkyl groups. Methods for making polyclonal antibodies are known in the art, and are described, for example, in Antibodies: A Laboratory Manual, Lane and Harlow (1988), incorporated by reference herein.

### Monoclonal Antibodies

A “monoclonal antibody” as used herein is intended to refer to a hybridoma-derived antibody (e.g., an antibody secreted by a hybridoma prepared by hybridoma technology, such as the standard Kohler and Milstein hybridoma methodology). For example, the monoclonal antibodies may be made using the hybridoma method first described by Kohler et al., Nature, 256:495 (1975), or may be made by recombinant DNA methods (U.S. Pat. No. 4,816,567). Thus, a hybridoma-derived dual-specificity antibody of the present disclosure is still referred to as a monoclonal antibody although it has antigenic specificity for more than a single antigen.

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

In a further embodiment, antibodies can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res., 21:2265-2266 (1993)). Thus, these techniques are viable alternatives to traditional monoclonal antibody hybridoma techniques for isolation of monoclonal antibodies.

Antibodies and antibody fragments may also be isolated from yeast and other eukaryotic cells with the use of expression libraries, as described in U.S. Pat. Nos. 6,423,538; 6,696,251; 6,699,658; 6,300,065; 6,399,763; and 6,114,147. Eukaryotic cells may be engineered to express library proteins, including from combinatorial antibody libraries, for display on the cell surface, allowing for selection of particular cells containing library clones for antibodies with affinity to select target molecules. After recovery from an isolated cell, the library clone coding for the antibody of interest can be expressed at high levels from a suitable mammalian cell line.

Additional methods for developing antibodies of interest include cell-free screening using nucleic acid display technology, as described in U.S. Pat. Nos. 7,195,880; 6,951,725; 7,078,197; 7,022,479, 6,518,018; 7,125,669; 6,846,655; 6,281,344; 6,207,446; 6,214,553; 6,258,558; 6,261,804; 6,429,300; 6,489,116; 6,436,665; 6,537,749; 6,602,685; 6,623,926; 6,416,950; 6,660,473; 6,312,927; 5,922,545; and 6,348,315. These methods can be used to transcribe a protein in vitro from a nucleic acid in such a way that the protein is physically associated or bound to the nucleic acid from which it originated. By selecting for an expressed protein with a target molecule, the nucleic acid that codes for the protein is also selected. In one variation on cell-free screening techniques, antibody sequences isolated from immune system cells can be isolated and partially randomized polymerase chain reaction mutagenesis techniques to increase antibody diversity. These partially randomized antibody genes are then expressed in a cell-free system, with concurrent physical association created between the nucleic acid and antibody.

The DNA also may be modified, for example, by substituting the coding sequence for human heavy- and light-chain constant domains in place of the homologous murine sequences (U.S. Pat. No. 4,816,567; Morrison, et al., Proc. Natl. Acad. Sci. USA, 81:6851 (1984)), or by covalently joining to the immunoglobulin coding sequence all or part of the coding sequence for a non-immunoglobulin polypeptide.

Typically such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

Chimeric or hybrid antibodies also may be prepared in vitro using known methods in synthetic protein chemistry, including those involving crosslinking agents. For example, immunotoxins may be constructed using a disulfide-exchange reaction or by forming a thioether bond. Examples of suitable reagents for this purpose include iminothiolate and methyl-4-mercaptobutyrimidate.

### Humanized Antibodies

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting non-human (e.g., rodent) CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some framework (FR) residues are substituted by residues from analogous sites in rodent antibodies. Additional references which describe the humanization process include Sims et al., J. Immunol., 151:2296 (1993); Chothia et al., J. Mol. Biol., 196:901 (1987); Carter et al., Proc. Natl. Acad. Sci. USA, 89:4285 (1992); Presta et al., J. Immunol., 151:2623 (1993); each of which is incorporated by reference herein.

### Human Antibodies

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993). Human antibodies can also be derived from phage-display libraries (Hoogenboom et al., J. Mol. Biol., 227:381 (1991); Marks et al., J. Mol. Biol., 222:581-597 (1991)).

In one embodiment, the formulation of the present disclosure comprises an antibody, or antigen-binding portion thereof, which binds human TNFα, including, for example, adalimumab (also referred to as Humira, adalimumab, or D2E7; AbbVie). In one embodiment, the antibody, or antigen-binding fragment thereof, dissociates from human TNFα with a IQ of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. Examples and methods for making human, neutralizing antibodies which have a high affinity for human TNFα, including sequences of the antibodies, are described in U.S. Pat. No. 6,090,382 (referred to as D2E7), incorporated herein by reference.

In one embodiment, the formulation of the present disclosure comprises an antibody, or antigen-binding portion thereof, which binds human IL-12, including, for example, J695 (U.S. Pat. No. 6,914,128) (AbbVie). J695 is a fully human monoclonal antibody designed to target and neutralize interleukin-12 and interleukin-23. Examples and methods for making human, neutralizing antibodies which have a high affinity for human IL-12, including sequences of the antibody, are described in U.S. Pat. No. 6,914,128, incorporated by reference herein.

In one embodiment, the formulation of the present disclosure comprises an anti-IL-13 antibody, or antigen-binding portion thereof, which is the antibody 13C5.5 (AbbVie) (see PCT/US2007/19660 (WO 08/127,271), incorporated by reference herein).

In one embodiment, the formulation of the present disclosure comprises an anti-RGMa antibody, or antigen-binding portion thereof, which is the antibody CPA4026 (AbbVie) (see US 2010/0028340, incorporated by reference herein. In one embodiment, the formulation of the present disclosure comprises an anti-IL-1α/IL-1β DVD-Ig (DVD12-1CHO), or antigen-binding portion thereof (AbbVie) (see US 2011/0280800, incorporated by reference herein).

In one embodiment, the formulation of the present disclosure comprises an anti-NGF antibody, or antigen-binding portion thereof, which is the antibody PG110 (AbbVie) (see US 2010/0278839, incorporated by reference herein.

In one embodiment, the formulation of the present disclosure comprises an anti-EGFR antibody, or antigen-binding portion thereof, which is the antibody 111-10 (AbbVie).

### Bispecific Antibodies

Bispecific antibodies (BsAbs) are antibodies that have binding specificities for at least two different epitopes. Such antibodies can be derived from full length antibodies or antibody fragments (e.g., F(ab′)₂ bispecific antibodies).

Methods for making bispecific antibodies are known in the art. Traditional production of full length bispecific antibodies is based on the coexpression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829 and in Traunecker et al., EMBO J., 10:3655-3659 (1991).

According to a different approach, antibody variable domains with the desired binding specificities (antibody-antigen combining sites) are fused to immunoglobulin constant domain sequences.

In an embodiment, the fusion is with an immunoglobulin heavy chain constant domain, comprising at least part of the hinge, CH2, and CH3 regions. In an embodiment, the first heavy-chain constant region (CH1) containing the site necessary for light chain binding is present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In an aspect of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690 published Mar. 3, 1994. For further details of generating bispecific antibodies see, for example, Suresh et al., Methods in Enzymology, 121:210 (1986).

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. The following techniques can also be used for the production of bivalent antibody fragments which are not necessarily bispecific. For example, Fab′ fragments recovered from E. coli can be chemically coupled in vitro to form bivalent antibodies. See, Shalaby et al., J. Exp. Med., 175:217-225 (1992).

Various techniques for making and isolating bivalent antibody fragments directly from recombinant cell culture have also been described. For example, bivalent heterodimers have been produced using leucine zippers. Kostelny et al., J. Immunol., 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. The “diabody” technology described by Hollinger et al., Proc. Natl. Acad. Sci. USA, 90:6444-6448 (1993) has provided an alternative mechanism for making bispecific/bivalent antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific/bivalent antibody fragments by the use of single-chain Fv (sFv) dimers has also been reported. See Gruber et al., J. Immunol., 152:5368 (1994).

In one embodiment, the formulation of the present disclosure comprises an antibody which is bispecific for IL-1 (including IL-1α and IL-1β). Examples and methods for making bispecific IL-1 antibodies can be found in U.S. Provisional Appln. No. 60/878,165, filed Dec. 29, 2006.

### Diafiltration/Ultrafiltration

Diafiltration/Ultrafiltration (also generally referred to herein as DF/UF) selectively utilizes permeable (porous) membrane filters to separate the components of solutions and suspensions based on their molecular size. A membrane retains molecules that are larger than the pores of the membrane while smaller molecules such as salts, solvents and water, which are permeable, freely pass through the membrane. The solution retained by the membrane is known as the concentrate or retentate. The solution that passes through the membrane is known as the filtrate or permeate. One parameter for selecting a membrane for concentration is its retention characteristics for the sample to be concentrated. As a general rule, the molecular weight cut-off (MWCO) of the membrane should be ⅓rd to ⅙th the molecular weight of the molecule to be retained. This is to assure complete retention. The closer the MWCO is to that of the sample, the greater the risk for some small product loss during concentration. Examples of membranes that can be used with methods of the present disclosure include Omega™ PES membrane (30 kDa MWCO, i.e. molecules larger than 30 kDa are retained by the membrane and molecules less than 30 kDa are allowed to pass to the filtrate side of the membrane) (Pall Corp., Port Washington, N.Y.); Millex®-GV Syringe Driven Filter Unit, PVDF 0.22 μm (Millipore Corp., Billerica, Mass.); Millex®-GP Syringe Driven Filter Unit, PES 0.22 μm; Sterivex® 0.22 μm Filter Unit (Millipore Corp., Billerica, Mass.); and Vivaspin concentrators (MWCO 10 kDa, PES; MWCO 3 kDa, PES) (Sartorius Corp., Edgewood, N.Y.). In order to prepare a low-ionic protein formulation of the present disclosure, the protein solution (which may be solubilized in a buffered formulation) is subjected to a DF/UF process, whereby water is used as a DF/UF medium. In an aspect, the DF/UF medium consists of water and does not include any other excipients.

Any water can be used in the DF/UF process of the present disclosure. In an embodiment, the water used is purified or deionized water. Types of water known in the art that may be used in the practice of the present disclosure include water for injection (WFI) (e.g., HyPure WFI Quality Water (HyClone), AQUA-NOVA® WFI (Aqua Nova)), UltraPure™ Water (Invitrogen), and distilled water (Invitrogen; Sigma-Aldrich).

There are two forms of DF/UF, including DF/UF in discontinuous mode and DF/UF in continuous mode. The methods of the present disclosure may be performed according to either mode.

Continuous DF/UF (also referred to as constant volume DF/UF) involves washing out the original buffer salts (or other low molecular weight species) in the retentate (sample or first protein solution) by adding water or a new buffer to the retentate at the same rate as filtrate is being generated. As a result, the retentate volume and product concentration does not change during the DF/UF process. The amount of salt removed is related to the filtrate volume generated, relative to the retentate volume. The filtrate volume generated is usually referred to in terms of “diafiltration volumes”. A single diafiltration volume (DV) is the volume of retentate when diafiltration is started. For continuous diafiltration, liquid is added at the same rate as filtrate is generated. When the volume of filtrate collected equals the starting retentate volume, 1 DV has been processed.

Discontinuous DF/UF includes two different methods, discontinuous sequential DF/UF and volume reduction discontinuous DF/UF. Discontinuous DF/UF by sequential dilution involves first diluting the sample (or first protein solution) with water to a predetermined volume. The diluted sample is then concentrated back to its original volume by UF. Discontinuous DF/UF by volume reduction involves first concentrating the sample to a predetermined volume, then diluting the sample back to its original volume with water or replacement buffer. As with continuous DF/UF, the process is repeated until the level of unwanted solutes, e.g., ionic excipients, are removed.

DF/UF may be performed in accordance with conventional techniques known in the art using water, e.g., WFI, as the DF/UF medium (e.g., Industrial Ultrafiltration Design and Application of Diafiltration Processes, Beaton & Klinkowski, J. Separ. Proc. Technol., 4(2) 1-10 (1983)). Examples of commercially available equipment for performing DF/UF include Millipore Labscale™ TFF System (Millipore), LV Centramate™ Lab Tangential Flow System (Pall Corporation), and the UniFlux System (GE Healthcare). For example, in an aspect, the Millipore Labscale™ Tangential Flow Filtration (TFF) system with a 500 mL reservoir is used to perform a method of the present disclosure to produce a diafiltered antibody solution. For exemplary equipment, solution and water volumes, number of process steps, and other parameters of particular embodiments of the present disclosure, see the Examples section below.

Alternative methods to diafiltration for buffer exchange where a protein is re-formulated into water in accordance with the present disclosure include dialysis and gel filtration, both of which are techniques known to those in the art. Dialysis requires filling a dialysis bag (membrane casing of defined porosity), tying off the bag, and placing the bag in a bath of water. Through diffusion, the concentration of salt in the bag will equilibrate with that in the bath, wherein large molecules, e.g., proteins that cannot diffuse through the bag remain in the bag. The greater the volume of the bath relative to the sample volume in the bags, the lower the equilibration concentration that can be reached. Generally, replacements of the bath water are required to completely remove all of the salt. Gel filtration is a non-adsorptive chromatography technique that separates molecules on the basis of molecular size. In gel filtration, large molecules, e.g., proteins, may be separated from smaller molecules, e.g., salts, by size exclusion.

In an aspect of the present disclosure, the first protein solution is subjected to a repeated volume exchange with the water, such that an aqueous formulation, which is essentially water and protein, is achieved. The diafiltration step may be performed any number of times, depending on the protein in solution, wherein one diafiltration step equals one total volume exchange. In one embodiment, the diafiltration process is performed 1, 2, 3, 4, 5, 6, 7, 8, 9, or up to as many times are deemed necessary to remove excipients, e.g., salts, from the first protein solution, such that the protein is dissolved essentially in water. A single round or step of diafiltration is achieved when a volume of water has been added to the retentate side that is equal to the starting volume of the protein solution.

In one embodiment, the protein solution is subjected to at least 2 diafiltration steps. In one embodiment, the diafiltration step or volume exchange with water may be repeated at least four times, and in an embodiment, repeated at least about five times. In one embodiment, the first protein solution is subjected to diafiltration with water until at least a six-fold volume exchange is achieved. In another embodiment, the first protein solution is subjected to diafiltration with water until at least an eight-fold volume exchange is achieved. Ranges intermediate to the above recited numbers, e.g., 4 to 6 or 5 to 7, are also intended to be part of this present disclosure. For example, ranges of values using a combination of any of the above recited values as upper and/or lower limits are intended to be included.

In an aspect, loss of protein to the filtrate side of an ultrafiltration membrane should be minimized. The risk of protein loss to the filtrate side of a particular membrane varies in relation to the size of the protein relative to the membrane's pore size, and the protein's concentration. With increases in protein concentration, risk of protein loss to the filtrate increases. For a particular membrane pore size, risk of protein loss is greater for a smaller protein that is close in size to the membrane's MWCO than it is for a larger protein. Thus, when performing DF/UF on a smaller protein, it may not be possible to achieve the same reduction in volume, as compared to performing DF/UF on a larger protein using the same membrane, without incurring unacceptable protein losses. In other words, as compared to the ultrafiltration of a solution of a smaller protein using the same equipment and membrane, a solution of a larger protein could be ultrafiltered to a smaller volume, with a concurrent higher concentration of protein in the solution. DF/UF procedures using a particular pore size membrane may require more process steps for a smaller protein than for a larger protein; a greater volume reduction and concentration for a larger protein permits larger volumes of water to be added back, leading to a larger dilution of the remaining buffer or excipient ingredients in the protein solution for that individual process step. Fewer process steps may therefore be needed to achieve a certain reduction in solutes for a larger protein than for a smaller one. A person with skill in the art would be able to calculate the amount of concentration possible with each process step and the number of overall process steps required to achieve a certain reduction in solutes, given the protein size and the pore size of the ultrafiltration device to be used in the procedure.

As a result of the diafiltration methods of the present disclosure, the concentration of non-protein solutes in the first protein solution is significantly reduced in the final aqueous formulation comprising essentially water and protein. For example, the aqueous formulation may have a final concentration of excipients which is at least 95% less than the first protein solution, and, in an embodiment, at least 99% less than the first protein solution. For example, in one embodiment, to dissolve a protein in WFI is a process that creates a theoretical final excipient concentration, reached by constant volume diafiltration with five diafiltration volumes, that is equal or approximate to Ci e⁻⁵=0.00674, i.e., an approximate 99.3% maximum excipient reduction. In one embodiment, a person with skill in the art may perform 6 volume exchanges during the last step of a commercial DF/UF with constant volume diafiltration, i.e., Ci would be Cᵢ·e⁻⁶=0.0025. This would provide an approximate 99.75% maximum theoretical excipient reduction. In another embodiment, a person with skill in the art may use 8 diafiltration volume exchanges to obtain a theoretical about 99.9% maximum excipient reduction.

The term “excipient-free” or “free of excipients” indicates that the formulation is essentially free of excipients. In one embodiment, excipient-free indicates buffer-free, salt-free, sugar-free, amino acid-free, surfactant-free, and/or polyol free. In one embodiment, the term “essentially free of excipients” indicates that the solution or formulation is at least 99% free of excipients. It should be noted, however, that in certain embodiments, a formulation may comprise a certain specified non-ionic excipient, e.g., sucrose or mannitol, and yet the formulation is otherwise excipient free. For example, a formulation may comprise water, a protein, and mannitol, wherein the formulation is otherwise excipient free. In another example, a formulation may comprise water, a protein, and polysorbate 80, wherein the formulation is otherwise excipient free. In yet another example, the formulation may comprise water, a protein, a sorbitol, and polysorbate 80, wherein the formulation is otherwise excipient free.

When water is used for diafiltering a first protein solution in accordance with the methods described herein, ionic excipients will be washed out, and, as a result, the conductivity of the diafiltered aqueous formulation is lower than the first protein solution. If an aqueous solution conducts electricity, then it must contain ions, as found with ionic excipients. A low conductivity measurement is therefore indicative that the aqueous formulation of the present disclosure has significantly reduced excipients, including ionic excipients.

Conductivity of a solution is measured according to methods known in the art. Conductivity meters and cells may be used to determine the conductivity of the aqueous formulation, and should be calibrated to a standard solution before use. Examples of conductivity meters available in the art include MYRON L Digital (Cole Parmer®), Conductometer (Metrohm AG), and Series 3105/3115 Integrated Conductivity Analyzers (Kemotron). In one embodiment, the aqueous formulation has a conductivity of less than 3 mS/cm. In another embodiment, the aqueous formulation has a conductivity of less than 2 mS/cm. In yet another embodiment, the aqueous formulation has a conductivity of less than 1 mS/cm. In one aspect of the present disclosure, the aqueous formulation has a conductivity of less than 0.5 mS/cm. Ranges intermediate to the above recited numbers, e.g., 1 to 3 mS/cm, are also intended to be encompassed by the present disclosure. For example, ranges of values using a combination of any of the above recited values as upper and/or lower limits are intended to be included. In addition, values that fall within the recited numbers are also included in the present disclosure, e.g., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 mS/cm and so forth.

An aspect of the present disclosure is that the diafiltered protein solution (solution obtained following the diafiltration process of the first protein solution) can be concentrated. By following this process, it has been discovered that high concentrations of protein are stable in water, for at least the duration of the processing. Concentration following diafiltration results in an aqueous formulation containing water and an increased protein concentration relative to the first protein solution. Thus, the present disclosure also includes diafiltering a protein solution using water as a diafiltration medium and subsequently concentrating the resulting aqueous solution. Concentration of the diafiltered protein solution may be achieved through means known in the art, including centrifugation. For example, following diafiltration, the water-based diafiltrated protein solution is subjected to a centrifugation process which serves to concentrate the protein via ultrafiltration into a high concentration formulation while maintaining the water-based solution. Means for concentrating a solution via centrifugation with ultrafiltration membranes and/or devices are known in the art, e.g., with Vivaspin centrifugal concentrators (Sartorius Corp. Edgewood, N.Y.).

The methods of the present disclosure provide a means of concentrating a protein at very high levels in water without the need for additional stabilizing agents. The concentration of the protein in the aqueous formulation obtained using the methods of the present disclosure can be any amount in accordance with the desired concentration. For example, the concentration of protein in an aqueous solution made according to the methods herein is at least about 10 μg/mL; at least about 1 mg/mL; at least about 10 mg/mL; at least about 20 mg/mL; at least about 50 mg/mL; at least about 75 mg/mL; at least about 100 mg/mL; at least about 125 mg/mL; at least about 150 mg/mL; at least about 175 mg/mL; at least about 200 mg/mL; at least about 220 mg/mL; at least about 250 mg/mL; at least about 300 mg/mL; or greater than about 300 mg/mL: Ranges intermediate to the above recited concentrations, e.g., at least about 80 mg/mL, at least about 125 mg/mL, at least about 195 mg/mL, and at least about 300 mg/mL, are also intended to be encompassed by the present disclosure. In addition, ranges of values using a combination of any of the above recited values (or values between the ranges described above) as upper and/or lower limits are intended to be included, e.g., 50 to 75 mg/mL, 60 to 80 mg/L, 75 to 100 mg/mL, 100 to 125 mg/mL, 110 to 125 mg/mL, and 126 to 200 mg/mL or more.

The methods of the present disclosure provide the advantage that the resulting formulation has a low percentage of protein aggregates, despite the high concentration of the aqueous protein formulation. In one embodiment, the aqueous formulations comprising water and a high concentration of a protein, e.g., antibodies, contains less than about 5% protein aggregates, even in the absence of a surfactant or other type of excipient. In one embodiment, the formulation comprises no more than about 5% aggregate protein; the formulation comprises no more than about 4% aggregate protein; the formulation comprises no more than about 3% aggregate protein; the formulation comprises no more than about 2% aggregate protein; or the formulation comprising no more than about 1% aggregate protein. In one embodiment, the formulation comprises at least about 92%, at least about 93%, at least about 94%, at least about 95%, at least about 96%, at least about 97%, at least about 98%, or at least about 99% monomer protein. Ranges intermediate to the above recited concentrations, e.g., at least about 94.7% monomer, no more than about 4.7% aggregate protein, are also intended to be part of this present disclosure. In addition, ranges of values using a combination of any of the above recited values as upper and/or lower limits are intended to be included.

Many protein-based pharmaceutical products need to be formulated at high concentrations. For example, antibody-based products increasingly tend to exceed 100 mg/mL in their Drug Product (DP) formulation to achieve appropriate efficacy and meet a typical patient usability requirement of a maximal about 1 mL injection volume. Accordingly, downstream processing steps, such as diafiltration into the final formulation buffer or ultrafiltration to increase the protein concentration, are also conducted at higher concentrations.

Classic thermodynamics predicts that intermolecular interactions can affect the partitioning of small solutes across a dialysis membrane, especially at higher protein concentrations, and models describing non-ideal dialysis equilibrium and the effects of intermolecular interactions are available (Tanford Physical chemistry or macromolecules. New York, John Wiley and Sons, Inc., p. 182, 1961; Tester and Modell Thermodynamics and its applications, 3.sup.rd ed. Upper Saddle River, NL, Prentice-Hall, 1997). In the absence of the availability of detailed thermodynamic data in the process development environment, which is necessary to apply these type of models, intermolecular interactions rarely are taken into account during the design of commercial DF/UF operations. Consequently, DP excipient concentrations may differ significantly from the concentration labeled. Several examples of this discrepancy in commercial and development products are published, e.g., chloride being up to 30% lower than labeled in an IL-1 receptor antagonist, histidine being 40% lower than labeled in a PEG-sTNF receptor, and acetate being up to 200% higher than labeled in a fusion conjugate protein (Stoner et al., J. Pharm. Sci., 93, 2332-2342 (2004)). There are several reasons why the actual DP may be different from the composition of the buffer the protein is diafiltered into, including the Donnan effect (Tombs and Peacocke (1974) Oxford; Clarendon Press), non-specific interactions (Arakawa and Timasheff, Arch. Biochem. Biophys., 224, 169-77 (1983); Timasheff, Annu. Rev. Biophys. Biomol. Struct., 22, 67-97 (1993)), and volume exclusion effects. Volume exclusion includes most protein partial specific volumes are between 0.7 and 0.8 mL/g. Thus, for a globular protein at 100 mg/mL, protein molecules occupy approx. 7.5% of the total solution volume. No significant intermolecular interactions assumed, this would translate to a solute molar concentration on the retentate side of the membrane that is 92.5% of the molar concentration on the permeate side of the membrane. This explains why basically all protein solution compositions necessarily change during ultrafiltration processing. For instance, at 40 mg/mL the protein molecules occupy approx. 3% of the total solution volume, and an ultrafiltration step increasing the concentration to 150 mg/mL will necessarily induce molar excipient concentrations to change by more than 8% (as protein at 150 mg/mL accounts for more than 11% of total solution volume). Ranges intermediate to the above recited percentages are also intended to be part of this present disclosure. In addition, ranges of values using a combination of any of the above recited values as upper and/or lower limits are intended to be included.

In accordance with the methods and compositions of the present disclosure, buffer composition changes during DF/UF operations can be circumvented by using pure water as diafiltration medium. By concentrating the protein about 20% more than the concentration desired in the final Bulk DS, excipients could subsequently be added, for instance, via highly concentrated excipient stock solutions and/or by spiking with various buffer solutions. Excipient concentrations, buffer concentrations and solution pH could then be highly likely to be close to the value as calculated.

The aqueous formulation of the present disclosure provides an advantage as a starting material, as it essentially contains no excipient. Any excipient(s) which is added to the formulation following the diafiltration in water can be accurately calculated, i.e., pre-existing concentrations of excipient(s) do not interfere with the calculation. Examples of pharmaceutically acceptable excipients are described in Remington's Pharmaceutical Sciences 16th edition, Osol, A. Ed. (1980), incorporated by reference herein. Thus, another aspect of the present disclosure includes using the aqueous formulation obtained through the methods described herein, for the preparation of a formulation, particularly a pharmaceutical formulation, having known concentrations of excipient(s), including non-ionic excipient(s) or ionic excipient(s). One aspect of the present disclosure includes an additional step where an excipient(s) is added to the aqueous formulation comprising water and protein. Thus, the methods of the present disclosure provide an aqueous formulation which is essentially free of excipients and may be used as a starting material for preparing formulations comprising water, proteins, and specific concentrations of excipients.

In one embodiment, the methods of the present disclosure may be used to add non-ionic excipients, e.g., sugars or non-ionic surfactants, such as polysorbates and poloxamers, to the formulation without changing the characteristics, e.g., protein concentration, hydrodynamic diameter of the protein, conductivity, etc.

Additional characteristics and advantages of aqueous formulations obtained using the above methods are described below. Exemplary protocols for performing the methods of the present disclosure are also described below in the Examples.

In an embodiment, different antibody process streams, 13C5.5, CPA4026, PG110, 111-10, or DVD12-1CHO were used to evaluate the feasibility of the approach of diafiltration into water along with addition of histidine stock solution into the concentrated retentate to formulate bulk drug substance (BDS) at a given target. Using the UF/DF methods disclosed herein the antibody product showed comparable quality, characteristics, and process yield as standard UF/DF operation with diafiltration into buffers such as histidine buffers. Using the methods of UF/DF provided herein enables rapid process development with minimal material needs, enhances process uniformity over wide range of molecules and allows better control of pH and formulation compositions.

For some antibody molecules, reducing the feed pH with acetic acid and/or citric acid prior to UF/DF processing or adding a low level of Tween into water for diafiltration effectively mitigated antibody aggregation and particle/turbidity formation during the buffer exchange and the protein concentration processes. Using the UF/DF methods disclosed herein improved material filterability, reduced filter area, and enhanced process yield and product quality.

The present disclosure provides an aqueous formulation comprising a protein and water which has a number of advantages over conventional formulations in the art, including stability of the protein in water without the requirement for additional excipients, increased concentrations of protein without the need for additional excipients to maintain solubility of the protein, and low osmolality. The formulations of the present disclosure also have advantageous storage properties, as the proteins in the formulation remain stable during storage. In one embodiment, formulations of the present disclosure include high concentrations of proteins such that the aqueous formulation does not show significant opalescence, aggregation, or precipitation.

The concentration of the aqueous formulation of the present disclosure is not limited by the protein size and the formulation may include any size range of proteins. Included within the scope of the present disclosure is an aqueous formulation comprising at least about 50 mg/mL and as much as 200 mg/mL or more of a protein, which may range in size from 5 kDa to 150 kDa or more. In one embodiment, the protein in the formulation of the present disclosure is at least about 15 kD in size, at least about 20 kD in size; at least about 47 kD in size; at least about 60 kD in size; at least about 80 kD in size; at least about 100 kD in size; at least about 120 kD in size; at least about 140 kD in size; at least about 160 kD in size; or greater than about 160 kD in size, or greater than about 200 kD in size. Ranges intermediate to the above recited sizes are also intended to be part of this present disclosure. In addition, ranges of values using a combination of any of the above recited values as upper and/or lower limits are intended to be included.

The aqueous formulation of the present disclosure may be characterized by the hydrodynamic diameter (Dₕ) of the proteins in solution. The hydrodynamic diameter of the protein in solution may be measured using dynamic light scattering (DLS), which is an established analytical method for determining the Dₕ of proteins. Typical values for monoclonal antibodies, e.g., IgG, are about 10 nm. Low-ionic formulations, like those described herein, may be characterized in that the Dₕ of the proteins are notably lower than protein formulations comprising ionic excipients.

In one embodiment, the Dₕ of the protein in the aqueous formulation is smaller relative to the Dₕ of the same protein in a buffered solution, irrespective of protein concentration. Thus, in certain embodiments, protein in an aqueous formulation made in accordance with the methods described herein, will have a Dₕ which is at least 25% less than the Dₕ of the protein in a buffered solution at the same given concentration. Examples of buffered solutions include, but are not limited to phosphate buffered saline (PBS). In certain embodiments, proteins in the aqueous formulation of the present disclosure have a Dₕ that is at least 50% less than the Dₕ of the protein in PBS in at the given concentration; at least 60% less than the Dₕ of the protein in PBS at the given concentration; at least 70% less than the Dₕ of the protein in PBS at the given concentration; or more than 70% less than the Dₕ of the protein in PBS at the given concentration. Ranges intermediate to the above recited percentages are also intended to be part of this present disclosure, e.g., 55%, 56%, 57%, 64%, 68%, and so forth. In addition, ranges of values using a combination of any of the above recited values as upper and/or lower limits are intended to be included, e.g., 50% to 80%.

Protein aggregation is a common problem in protein solutions, and often results from increased concentration of the protein. The instant present disclosure provides a means for achieving a high concentration, low protein aggregation formulation. In one embodiment, formulations of the present disclosure do not rely on a buffering system and excipients, including surfactants, to keep proteins in the formulation soluble and from aggregating. Formulations of the present disclosure can be advantageous for therapeutic purposes, as they are high in protein concentration and water-based, not relying on other agents to achieve high, stable concentrations of proteins in solution.

The majority of biologic products (including antibodies) are subject to numerous degradative processes which frequently arise from non-enzymatic reactions in solution. These reactions may have a long-term impact on product stability, safety and efficacy. These instabilities can be retarded, if not eliminated, by storage of product at subzero temperatures, thus gaining a tremendous advantage for the manufacturer in terms of flexibility and availability of supplies over the product life-cycle. Although freezing is often the safest and most reliable method of biologics product storage, it has inherent risks. Freezing can induce stress in proteins through cold denaturation, by introducing ice-liquid interfaces, and by freeze-concentration (cryoconcentration) of solutes when the water crystallizes.

Cryoconcentration is a process in which a flat, uncontrolled moving ice front is formed during freezing that excludes solute molecules (small molecules such as sucrose, salts, and other excipients typically used in protein formulation, or macromolecules such as proteins), leading to zones in which proteins may be found at relatively high concentration in the presence of other solutes at concentrations which may potentially lead to local pH or ionic concentration extremes. For most proteins, these conditions can lead to denaturation and in some cases, protein and solute precipitation. Since buffer salts and other solutes are also concentrated under such conditions, these components may reach concentrations high enough to lead to pH and/or redox changes in zones within the frozen mass. The pH shifts observed as a consequence of buffer salt crystallization (e.g., phosphates) in the solutions during freezing can span several pH units, which may impact protein stability.

Concentrated solutes may also lead to a depression of the freezing point to an extent where the solutes may not be frozen at all, and proteins will exist within a solution under these adverse conditions. Often, rapid cooling may be applied to reduce the time period the protein is exposed to these undesired conditions. However, rapid freezing can induce a large-area ice-water interface, whereas slow cooling induces smaller interface areas. For instance, rapid cooling of six model proteins during one freeze/thaw step was shown to reveal a denaturation effect greater than 10 cycles of slow cooling, demonstrating the great destabilization potential of hydrophobic ice surface-induced denaturation.

The aqueous formulation of the present disclosure has advantageous stability and storage properties. Stability of the aqueous formulation is not dependent on the form of storage, and includes, but is not limited to, formulations which are frozen, lyophilized, or spray-dried. Stability can be measured at a selected temperature for a selected time period. In one aspect of the present disclosure, the protein in the aqueous formulations is stable in a liquid form for at least 3 months; at least 4 months, at least 5 months; at least 6 months; at least 12 months. Ranges intermediate to the above recited time periods are also intended to be part of this present disclosure, e.g., 9 months, and so forth. In addition, ranges of values using a combination of any of the above recited values as upper and/or lower limits are intended to be included. The formulation is stable at room temperature (about 30° C.) or at 40° C. for at least 1 month and/or stable at about 2-8° C. for at least 1 year, and/or stable at about 2-8° C. for at least 2 years. Furthermore, the formulation is stable following freezing (to, e.g., −80° C.) and thawing of the formulation, hereinafter referred to as a “freeze/thaw cycle.”

Stability of a protein can be also be defined as the ability to remain biologically active. A protein “retains its biological activity” in a pharmaceutical formulation, if the protein in a pharmaceutical formulation is biologically active upon administration to a subject. For example, biological activity of an antibody is retained if the biological activity of the antibody in the pharmaceutical formulation is within about 30%, about 20%, or about 10% (within the errors of the assay) of the biological activity exhibited at the time the pharmaceutical formulation was prepared (e.g., as determined in an antigen binding assay).

Stability of a protein in an aqueous formulation may also be defined as the percentage of monomer, aggregate, or fragment, or combinations thereof, of the protein in the formulation. A protein “retains its physical stability” in a formulation if it shows substantially no signs of aggregation, precipitation and/or denaturation upon visual examination of color and/or clarity, or as measured by UV light scattering or by size exclusion chromatography. In one aspect of the present disclosure, a stable aqueous formulation is a formulation having less than about 10% to less than about 5% of the protein being present as aggregate in the formulation.

Another characteristic of the aqueous formulation of the present disclosure is that, in some instances, diafiltering a protein using water results in an aqueous formulation having improved viscosity features in comparison to the first protein solution (i.e., the viscosity of the diafiltered protein solution is reduced in comparison to the first protein solution.) A person with skill in the art will recognize that multiple methods for measuring viscosity can be used in the preparation of formulations in various embodiments of the present disclosure. For example, kinematic viscosity data (cSt) may be generated using capillaries. In other embodiments, dynamic viscosity data is stated, either alone or with other viscosity data. The dynamic viscosity data may be generated by multiplying the kinematic viscosity data by the density.

In one embodiment, the present disclosure also provides a method for adjusting a certain characteristic, such as the osmolality and/or viscosity, as desired in high protein concentration-water solutions, by adding non-ionic excipients, such as mannitol, without changing other desired features, such as non-opalescence. As such, it is within the scope of the present disclosure to include formulations which are water-based and have high concentrations of protein, where, either during or following the transfer of the protein to water or during the course of the diafiltration, excipients are added which improve, for example, the osmolality or viscosity features of the formulation. Thus, it is also within the scope of the present disclosure that such non-ionic excipients could be added during the process of the transfer of the protein into the final low ionic formulation. Examples of non-ionizable excipients which may be added to the aqueous formulation of the present disclosure for altering desired characteristics of the formulation include, but are not limited to, mannitol, sorbitol, a non-ionic surfactant (e.g., polysorbate 20, polysorbate 40, polysorbate 60 or polysorbate 80), sucrose, trehalose, raffinose, and maltose.

The formulation herein may also contain more than one protein. With respect to pharmaceutical formulations, an additional, distinct, protein may be added as necessary for the particular indication being treated. In an embodiment, the distinct protein's complementary activities do not adversely affect the other protein. For example, it may be desirable to provide two or more antibodies which bind to TNF or IL-12 in a single formulation. Furthermore, anti-TNF or anti-IL-12 antibodies may be combined in the one formulation. Such proteins are suitably present in combination in amounts that are effective for the purpose intended.

Examples of proteins that may be included in the aqueous formulation include antibodies, or antigen-binding fragments thereof. Examples of different types of antibodies, or antigen-binding fragments thereof, that may be used in the present disclosure include, but are not limited to, a chimeric antibody, a human antibody, a humanized antibody, and a domain antibody (dAb).

In one embodiment, the antibody used in the methods and compositions of the present disclosure is an anti-TNFα antibody, or antigen-binding portion thereof, or an anti-IL-12 antibody, or antigen binding portion thereof.

Additional examples of an antibody or a DVD-Ig, or antigen-binding fragment thereof, that may be used in the present disclosure includes, but is not limited to, ID4.7 (anti-IL-12/anti-IL-23; AbbVie), 2.5 (E)mg1 (anti-IL-18; AbbVie), 13C5.5 (anti-II-13; AbbVie), J695 (anti-IL-12; AbbVie), Afelimomab (Fab 2 anti-TNF; AbbVie), Humira (adalimumab (D2E7); AbbVie), 13C5.5, CPA4026, PG110, 111-10 (AbbVie), DVD12-1CHO (AbbVie), Campath (Alemtuzumab), CEA-Scan Arcitumomab (fab fragment), Erbitux (Cetuximab), Herceptin (Trastuzumab), Myoscint (Imciromab Pentetate), ProstaScint (Capromab Pendetide), Remicade (Infliximab), ReoPro (Abciximab), Rituxan (Rituximab), Simulect (Basiliximab), Synagis (Palivizumab), Verluma (Nofetumomab), Xolair (Omalizumab), Zenapax (Daclizumab), Zevalin (Ibritumomab Tiuxetan), Orthoclone OKT3 (Muromonab-CD3), Panorex (Edrecolomab), and Mylotarg (Gemtuzumab ozogamicin) golimumab (Centocor), Cimzia (Certolizumab pegol), Saris (Eculizumab), CNTO 1275 (ustekinumab), Vectibix (panitumumab), Bexxar (tositumomab and I.sup.131 tositumomab), and Avastin (bevacizumab).

In one alternative, the protein is a therapeutic protein, including, but not limited to, Pulmozyme (Dornase alfa), Regranex (Becaplermin), Activase (Alteplase), Aldurazyme (Laronidase), Amevive (Alefacept), Aranesp (Darbepoetin alfa), Becaplermin Concentrate, Betaseron (Interferon beta-1b), BOTOX (Botulinum Toxin Type A), Elitek (Rasburicase), Elspar (Asparaginase), Epogen (Epoetin alfa), Enbrel (Etanercept), Fabrazyme (Agalsidase beta), Infergen (Interferon alfacon-1), Intron A (Interferon alfa-2a), Kineret (Anakinra), MYOBLOC (Botulinum Toxin Type B), Neulasta (Pegfilgrastim), Neumega (Oprelvekin), Neupogen (Filgrastim), Ontak (Denileukin diftitox), PEGASYS (Peginterferon alfa-2a), Proleukin (Aldesleukin), Pulmozyme (Dornase alfa), Rebif (Interferon beta-1a), Regranex (Becaplermin), Retavase (Reteplase), Roferon-A (Interferon alfa-2), TNKase (Tenecteplase), and Xigris (Drotrecogin alfa), Arcalyst (Rilonacept), NPlate (Romiplostim), Mircera (methoxypolyethylene glycol-epoetin beta), Cinryze (C1 esterase inhibitor), Elaprase (idursulfase), Myozyme (alglucosidase alfa), Orencia (abatacept), Naglazyme (galsulfase), Kepivance (palifermin) and Actimmune (interferon gamma-1b).

Other examples of proteins which may be included in the methods and compositions described herein, include mammalian proteins, including recombinant proteins thereof, such as, e.g., growth hormone, including human growth hormone and bovine growth hormone; growth hormone releasing factor; parathyroid hormone; thyroid stimulating hormone; lipoproteins; α-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or tissue-type plasminogen activator (t-PA); bombazine; thrombin; tumor necrosis factor-α and -β enkephalinase; RANTES (regulated on activation normally T-cell expressed and secreted); human macrophage inflammatory protein (MIP-1-α); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGFα and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins; CD proteins such as CD3, CD4, CD8, CD19 and CD20; erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

The formulations of the present disclosure may be used both therapeutically, i.e., in vivo, or as reagents for in vitro or in situ purposes.

### Therapeutic Uses

The methods of the present disclosure may also be used to make an aqueous formulation having characteristics which are advantageous for therapeutic use. The aqueous formulation may be used as a pharmaceutical formulation to treat a disorder in a subject.

In an embodiment, formulations and methods of the present disclosure may be used to treat any disorder for which the therapeutic protein is appropriate for treating. A “disorder” is any condition that would benefit from treatment with the protein. This includes chronic and acute disorders or diseases including those pathological conditions which predispose the mammal to the disorder in question. In the case of an anti-TNFα antibody, a therapeutically effective amount of the antibody may be administered to treat an autoimmune disease, such as rheumatoid arthritis or juvenile idiopathic arthritis, inflammatory arthritis, such as psoriatic arthritis or osteoarthritis, an intestinal disorder, such as Crohn's disease or ulcerative colitis, a spondyloarthropathy, such as ankylosing spondylitis, or a skin disorder, such as psoriasis. In the case of an anti-IL-12 antibody, a therapeutically effective amount of the antibody may be administered to treat a neurological disorder, such as multiple sclerosis, a skin disorder, such as psoriasis, inflammatory arthritis, such as psoriatic arthritis, or a chronic inflammatory disorder, such as sarcoidosis. Other examples of disorders in which the formulation of the present disclosure may be used to treat include blood disorders, such as neutropenia, leukemia, or lymphoma, central and peripheral neurological disorders, chronic and acute pain, and cancer, including lung cancer, breast cancer, prostate cancer, brain cancer, stomach cancer, and colon cancer.

The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the present disclosure, the subject is a human.

The term “treatment” refers to both therapeutic treatment and prophylactic or preventative measures. Those in need of treatment include those already with the disorder, as well as those in which the disorder is to be prevented.

The aqueous formulation may be administered to a mammal, including a human, in need of treatment in accordance with known methods of administration. Examples of methods of administration include intravenous administration, such as a bolus or by continuous infusion over a period of time, intramuscular, intraperitoneal, intracerobrospinal, subcutaneous, intra-articular, intrasynovial, intrathecal, intradermal, transdermal, oral, topical, or inhalation administration.

In one embodiment, the aqueous formulation is administered to the mammal by subcutaneous administration. For such purposes, the formulation may be injected using a syringe, as well as other devices including injection devices (e.g., the Inject-ease and Genject devices); injector pens (such as the GenPen); needleless devices (e.g., MediJector and Biojectorr 2000); and subcutaneous patch delivery systems. In one embodiment, the device, e.g., a syringe, autoinjector pen, contains a needle with a gauge ranging in size from 25 G or smaller in diameter. In one embodiment, the needle gauge ranges in size from 25G to 33 G (including ranges intermediate thereto, e.g., 25sG, 26, 26sG, 27G, 28G, 29G, 30G, 31G, 32G, and 33G). In an aspect, the smallest needle diameter and appropriate length is chosen in accordance with the viscosity characteristics of the formulation and the device used to deliver the formulation of the present disclosure.

The methods/compositions of the present disclosure provide large concentrations of a protein in a solution which may be ideal for administering the protein to a subject using a needleless device. Such a device allows for dispersion of the protein throughout the tissue of a subject without the need for an injection by a needle. Examples of needleless devices include, but are not limited to, Biojectorr 2000 (Bioject Medical Technologies), Cool Click (Bioject Medical Technologies), Iject (Bioject Medical Technologies), Vitajet 3 (Bioject Medical Technologies), Mhi500 (The Medical House PLC), Injex 30 (INJEX—Equidyne Systems), Injex 50 (INJEX—Equidyne Systems), Injex 100 (INJEX-Equidyne Systems), Jet Syringe (INJEX—Equidyne Systems), Jetinjector (Becton-Dickinson), J-Tip (National Medical Devices, Inc.), Medi-Jector VISION (Antares Pharma), MED-JET (MIT Canada, Inc.), DermoJet (Akra Dermojet), Sonoprep (Sontra Medical Corp.), PenJet (PenJet Corp.), MicroPor (Altea Therapeutics), Zeneo (Crossject Medical Technology), Mini-Ject (Valeritas Inc.), ImplaJect (Caretek Medical LTD), Intraject (Aradigm), and Serojet (Bioject Medical Technologies).

Also included in the present disclosure are delivery devices that house the aqueous formulation. Examples of such devices include, but are not limited to, a syringe, a pen (such as an autoinjector pen), an implant, an inhalation device, a needleless device, and a patch. An example of an autoinjection pen is described in U.S. application Ser. No. 11/824,516, filed Jun. 29, 2007.

The present disclosure also includes methods of delivering the formulations of the present disclosure by inhalation and inhalation devices containing said formulation for such delivery. In one embodiment, the aqueous formulation is administered to a subject via inhalation using a nebulizer or liquid inhaler. Generally, nebulizers use compressed air to deliver medicine as wet aerosol or mist for inhalation, and, therefore, require that the drug be soluble in water. Types of nebulizers include jet nebulizers (air-jet nebulizers and liquid-jet nebulizers) and ultrasonic nebulizers.

The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. The protein is suitably administered to the patient at one time or over a series of treatments and may be administered to the patient at any time from diagnosis onwards. The protein may be administered as the sole treatment or in conjunction with other drugs or therapies useful in treating the condition in question.

The methods disclosed herein and formulations prepared by methods of the present disclosure overcome the common problem of protein aggregation often associated with high concentrations of protein, and, therefore, provide a new means by which high levels of a therapeutic protein may be administered to a patient. The high concentration formulation of the present disclosure provides an advantage in dosing where a higher dose may be administered to a subject using a volume which is equal to or less than the formulation for standard treatment. Standard treatment for a therapeutic protein is described on the label provided by the manufacturer of the protein. For example, in accordance with the label provided by the manufacturer, infliximab is administered for the treatment of rheumatoid arthritis by reconstituting lyophilized protein to a concentration of 10 mg/mL. The formulation of the present disclosure may comprise a high concentration of infliximab, where a high concentration would include a concentration higher than the standard 10 mg/mL. In another example, in accordance with the label provided by the manufacturer, Xolair (omalizumab) is administered for the treatment of asthma by reconstituting lyophilized protein to a concentration of 125 mg/mL. In this instance, the high concentration formulation of the present disclosure would include a concentration of the antibody omalizumab which is greater than the standard 125 mg/mL.

Thus, in one embodiment, the formulation of the present disclosure comprises a high concentration which is at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 175%, at least about 200%, at least about 225%, at least about 250%, at least about 275%, at least about 300%, at least about 325%, at least about 350%, at least about 375%, at least about 400%, and so forth, greater than the concentration of a therapeutic protein in a known, standard formulation.

In another embodiment, the formulation of the present disclosure comprises a high concentration which is at least about 2 times greater than, at least about 3 times greater than, at least about 4 times greater than, at least about 5 times greater than, at least about 6 times greater than, at least about 7 times greater than, at least about 8 times greater than, at least about 9 times greater than, at least about 10 times greater than and so forth, the concentration of a therapeutic protein in a known, standard formulation.

Characteristics of the aqueous formulation may be improved for therapeutic use. For example, the viscosity of an antibody formulation may be improved by subjecting an antibody protein solution to diafiltration using water without excipients as the diafiltration medium. As described above in excipients, those which improve viscosity may be added back to the aqueous formulation such that the final concentration of excipient is known and the specific characteristic of the formulation is improved for the specified use. For example, one of skill in the art will recognize that the desired viscosity of a pharmaceutical formulation is dependent on the mode by which the formulation is being delivered, e.g., injected, inhaled, dermal absorption, and so forth. Often the desired viscosity balances the comfort of the subject in receiving the formulation and the dose of the protein in the formulation needed to have a therapeutic effect. For example, generally acceptable levels of viscosity for formulations being injected are viscosity levels of less than about 100 mPas, or less than about 75 mPas, or less than about 50 mPas. As such, viscosity of the aqueous formulation may be acceptable for therapeutic use, or may require addition of an excipient(s) to improve the desired characteristic.

### Non-Therapeutic Uses

The aqueous formulation of the present disclosure may also be used for non-therapeutic uses, i.e., in vitro purposes.

Aqueous formulations as well as methods disclosed herein may be used for diagnostic or experimental methods in medicine and biotechnology, including, but not limited to, use in genomics, proteomics, bioinformatics, cell culture, plant biology, and cell biology. For example, aqueous formulations described herein may be used to provide a protein needed as a molecular probe in a labeling and detecting methods. An additional use for the formulations described herein is to provide supplements for cell culture reagents, including cell growth and protein production for manufacturing purposes.

### Articles of Manufacture

In another embodiment of the present disclosure, an article of manufacture is provided which contains the aqueous formulation of the present disclosure and provides instructions for its use. The article of manufacture comprises a container. Suitable containers include, for example, bottles, vials (e.g., dual chamber vials), syringes (such as dual chamber syringes), autoinjector pen containing a syringe, and test tubes. The container may be formed from a variety of materials such as glass, plastic or polycarbonate. The container holds the aqueous formulation and the label on, or associated with, the container may indicate directions for use. For example, the label may indicate that the formulation is useful or intended for subcutaneous administration. The container holding the formulation may be a multi-use vial, which allows for repeat administrations (e.g., from 2-6 administrations) of the aqueous formulation. The article of manufacture may further comprise a second container. The article of manufacture may further include other materials desirable from a commercial and user standpoint, including other buffers, diluents, filters, needles, syringes, and package inserts with instructions for use.

The contents of all references, patents and published patent applications cited throughout this application are incorporated herein by reference.

The present disclosure is further illustrated by the following examples which should not be construed as limiting.

### EXAMPLES

The following examples describe experiments relating to methods disclosed herein. Drug substance or “DS” represents the active pharmaceutical ingredient and generally refers to a therapeutic protein in a common bulk solution.

In the examples below, a Pellicon XL Biomax 50 cm² membrane (Millipore) with a 30 kD MWCO or with a 50 kD MWCO was used for mAb or DVD-Ig preparations, respectively. Feed streams of 13C5.5, CPA4026, DVD12-1CHO, PG110 and 111-10 were first concentrated to a given concentration, diafiltered against water or a histidine buffer, and then concentrated to a final target concentration. Similar pressure and flow rates were used for both sets of conditions. During diafiltration, the retentate was analyzed for turbidity and conductivity. The antibodies which were diafiltered into water were formulated with 200 mM histidine at a selected pH to achieve the targeted histidine concentration of 15 mM and the targeted pH for the respective DS. The formulated bulk drug substance samples were measured for final pH, conductivity, turbidity, concentrations and aggregate/monomer levels by size exclusion chromatography (SEC). The samples were also held at 4° C. for 2 to 4 weeks and analyzed again by SEC to assess molecule stability in the respective formulation. In some cases, the levels of sub-visible particles in the DS were also measured using a micro-flow imaging technique (Brightwell, DPA 4200).

### Example 1

### 13C5.5

A 13C5.5 in-process feedstream, which was prepared from a hydrophobic interaction chromatography (HIC) polishing step and at about pH 7, was used in the following three UF/DF experiments. In two of the runs the concentrated feed was diafiltered against water while in the third run it was diafiltered against a diafiltration buffer having 23 mM histidine at pH 5.6. In each case, the HIC eluate was concentrated to a target of about 50 g/L, diafiltered against 8 diavolumes of the appropriate buffer, and then concentrated to a target of 180 g/L. The concentrate was then collected, the UF system rinsed with water (when diafiltered against water) or 15 mM histidine, pH 5.6 (when diafiltered against histidine), and the rinsate was added to the concentrate. The target final concentration was 140±20 g/L. After the experiment of diafiltration against water, the concentrated product was formulated by adding 200 mM histidine, pH 5.4 buffer at a volume ratio of 1:12.3 to meet the final 15 mM histidine concentration target.

As shown in Table 1, the experiment with diafiltration into water resulted in a yield of 98% versus 97% for the histidine diafiltered process. The final DS pH was within the target pH range of 5.3-6.3, and the conductivity and turbidity were very similar between the two diafiltration conditions. Relative to the 15 mM concentration target, the histidine level in the DS samples was 14.9 mM for material diafiltered against water and 16.6 mM for material diafiltered against histidine. Clearly, the former approach using water as the diafiltration medium followed by histidine spike showed better control of this excipient level in the final product.

TABLE 1
Comparison of 13C5.5 bulk drug substance (BDS) formulated by
different methods

| | Diafiltration | |
|---|---|---|
| Parameter | into water | Diafiltration into histidine |
| pH | 6.1 | 6.2 |
| Conductivity, mS/cm | 1.6 | 1.5 |
| Histidine Conc., mM | 14.9 | 16.6 |
| BDS Conc., g/L | 130 | 114 |
| Yield, % | 98 | 97 |

Assessment of product quality following the different UF/DF operations was monitored by SEC and turbidity analysis. The SEC profile of the bulk drug substance was similar for both diafiltration conditions and was within assay variability of the feed solution (see Table 2).

TABLE 2
Aggregates/monomer levels in 13C5.5 feed and BDS formulated from
different processes

| | @ Day 0 | | | After 2 week @ 4° C. | | |
|---|---|---|---|---|---|---|
| | Monomer | HMW | LMW | Monomer | HMW | LMW |
| Sample | % | % | % | % | % | % |
| UF/DF Feed | 98.1 | 1.8 | 0.1 | | N.D. | |
| BDS (diafiltered | 97.8 | 2.1 | 0.1 | 97.5 | 2.3 | 0.2 |
| into water) | | | | | | |
| BDS (diafiltered | 97.8 | 2.1 | 0.2 | 97.5 | 2.3 | 0.2 |
| into histidine) | | | | | | |

The normalized turbidity (i.e. measured NTU/protein concentration) data are tabulated in Table 3. The comparable turbidity values at various diavolumes suggest comparable molecule stability during these two processes. Overall, the data indicate that 13C5.5 can be diafiltered in water with no impact on product quality and process recovery, and the final product stability is comparable to that obtained from the standard histidine-diafiltered process.

TABLE 3
Normalized turbidity for 13C5.5 during diafiltration against water and
histidine buffer

| | Normalized Retentate Turbidity (NTU per g/L MAb) | | |
|---|---|---|---|
| | DF into | DF into | |
| Diavolume | water (Run 1) | water (Run 2) | DF into histidine buffer |
| 0 | 0.60 | 0.47 | 0.60 |
| 1 | 0.44 | 0.54 | 0.58 |
| 2 | 0.55 | 0.64 | 0.72 |
| 3 | 0.67 | 0.75 | 0.85 |
| 4 | 0.80 | 0.93 | 0.85 |
| 5 | 0.90 | 1.01 | 0.92 |
| 6 | 0.89 | 1.07 | 0.85 |
| 7 | 0.82 | 1.08 | 0.88 |
| 8 | 1.08 | 1.16 | 1.43 |

### Example 2

### CPA4026

CPA4026 in-process anion exchange (AEX) flow-through eluate at about pH 6.5 was used as the feed in the UF/DF experiments. In each experiment the feed was concentrated to a target of 50 g/L, diafiltered against 8 diavolumes of water or 23 mM histidine pH 5.6 buffer, and then concentrated to a target of 180 g/L. The concentrated retentate was collected, the UF system rinsed with water (when diafiltered against water) or 15 mM hisitidine, pH 5.6 (when diafiltered against histidine), and the rinsate was added to the concentrate. The target final concentration was 125±15 g/L. After the experiment of diafiltration against water, the concentrated product was formulated by adding 200 mM histidine, pH 5.4 buffer at a volume ratio of 1:12.3 to meet the final 15 mM histidine concentration target.

Table 4 and 5 compare the BDS attributes for CPA4026 from the two UF/DF processes. The pH, conductivity, and protein concentrations were all within expected ranges for both processes. Evaluation of the histidine content showed improved control in the retentate that was spiked with a stock solution rather than retentate that was diafiltered against histidine (which was designed to account for the “Donnon effect”). The small difference in the SEC profiles between the water-diafiltered and the histidine-diafiltered runs (Table 5) is within the typical experimental variations for this molecule. In addition, the final formulated DS obtained from the water-diafiltered run remained stable over a 3 week hold period with little changes in the aggregate/monomer levels.

TABLE 4
Comparison of CPA4026 BDS formulated by different methods

| Parameter | Diafiltration in water | Diafiltration in histidine |
|---|---|---|
| pH | 6.0 | 5.6 |
| Conductivity, mS/cm | 1.0 | 1.9 |
| Histidine Conc., mM | 14.2 | 16.9 |
| BDS Conc., g/L | 127 | 138 |
| Yield, % | 97 | 88 |

TABLE 5
Aggregates/monomer levels in CPA4026 feed and BDS formulated
from different processes

| | @ Day 0 | | | After 3 week @ 4° C. | | |
|---|---|---|---|---|---|---|
| | Monomer | HMW | LMW | Monomer | HMW | LMW |
| Sample | % | % | % | % | % | % |
| UF/DF Feed | 97.7 | 1.7 | 0.6 | N.D. | N.D. | N.D. |
| BDS (diafiltered | 97.0 | 2.2 | 0.8 | 96.8 | 2.3 | 0.9 |
| into water) | | | | | | |
| BDS (diafiltered | 97.5 | 1.8 | 0.7 | N.D. | N.D. | N.D. |
| into histidine) | | | | | | |

In contrast to the SEC data, the turbidity of retentate in histidine-diafiltered experiments was significantly higher than the run of diafiltration against water (Table 6). Overall, the data suggest that diafilteration of CPA4026 against water generates similar product quality without affecting process recovery as compared to diafiltration again a buffered solution.

TABLE 6
Normalized turbidity for CPA4026 during diafiltration against water
and histidine buffer

Normalized Retentate Turbidity
(NTU per g/L MAb)

| Diavolume | DF into water | DF into histidine buffer |
|---|---|---|
| 0 | 0.68 | N.D. |
| 1 | 0.87 | 1.5 |
| 2 | 0.96 | 1.6 |
| 3 | 0.87 | 2.0 |
| 4 | 0.78 | 2.0 |
| 5 | 0.91 | 2.1 |
| 6 | 0.89 | 3.5 |
| 7 | 0.88 | 3.7 |
| 8 | 0.88 | 3.9 |

### Example 3

### DVD12-1CHO

DVD12-1CHO was also evaluated for compatibility to diafiltration against water to generate a high protein concentration pharmaceutical. A thawed DVD12-1CHO AEX flow-through eluate at about pH 8 was first concentrated to about 50 g/L, diafiltered with 6 diavolumes of water, and then concentrated to a target of 110 g/L. The concentrate was collected, the UF system rinsed with water, and the rinsate was added to concentrate. The target final concentration was 80±10 g/L. After the experiment of diafiltration against water, the concentrated product was formulated by adding 200 mM histidine, pH 5.4 buffer at a volume ratio of 1:12.3 to meet the final 15 mM histidine concentration target. The results from the water diafiltration experiment was performed and compared with previous manufacturing data for the DS generated from histidine-diafiltered process. Tables 7 and 8 summarize the DS attributes from the two diafiltration-concentration processes. Clearly, the BDS pH values from both processes were comparable. The measured conductivity and turbidity of water-diafiltered DS were well within typically observed ranges for an antibody. The histidine concentration of DS obtained from water-diafiltration process was 14.8 mM. This is in contrast to the preparation that did not use the water-diafiltration process but instead was diafiltered with 15 mM histidine, and wherein the final histidine concentration was 12.2 mM. Hence, the water-diafiltered DVD12-1CHO followed by histidine addition allows better control of its concentration in the final drug product. The SEC profile of the water-diafiltered BDS is similar to that of the feed material (Table 8).

TABLE 7
Comparison of DVD12-1CHO BDS formulated by different methods

| | Diafiltration | Diafiltration into histidine |
|---|---|---|
| Parameter | into water | 2011 GMP Campaign |
| pH | 6.3 | 6.3 ± 0.3 |
| Conductivity, mS/cm | 1.2 | N.D. |
| Histidine Conc., mM | 14.8 | 12.2 |
| BDS Conc., g/L | 88 | 84 ± 6 |
| Yield, % | 97 | 101 ± 4 |

TABLE 8
Aggregates/monomer levels in DVD12-1CHO feed and BDS
formulated from different processes

| | @ Day 0 | | | After 2 week @ 4° C. | | |
|---|---|---|---|---|---|---|
| | Monomer | HMW | LMW | Monomer | HMW | LMW |
| Sample | % | % | % | % | % | % |
| UF/DF Feed | 96.9 | 1.4 | 1.7 | | N.D. | |
| BDS (diafiltered | 96.6 | 1.7 | 1.7 | 96.7 | 1.6 | 1.7 |
| into water) | | | | | | |

Table 9 shows the normalized turbidity values of the diafiltration retentate at each diavolume. The levels of turbidity formation during the course of diafiltration are similar to that for the other molecules.

Overall, DVD12-1CHO can be diafiltered in water with little to no impact on pH, conductivity, yield, aggregation or turbidity. Once it is formulated, this DVD-Ig molecule remains stable during extended holding/storage periods.

TABLE 9
Normalized turbidity for DVD12-1CHO during diafiltration against water

Normalized Retentate Turbidity
(NTU per g/L MAb)

| Diavolume | DF into water | DF into histidine buffer |
|---|---|---|
| 0 | 0.67 | N.D. |
| 1 | 0.80 | N.D. |
| 2 | 0.96 | N.D. |
| 3 | 1.12 | N.D. |
| 4 | 1.29 | N.D. |
| 5 | 1.59 | N.D. |
| 6 | 1.86 | N.D. |

### Example 4

### PG110

PG110 in-process material obtained from a CHT hydroxyapatite polishing step at about pH 7 was used as the feed for the UF/DF experiments described below. This feed material was either adjusted to pH 5.2 with 2 M acetic acid or left unadjusted before diafiltered into water or histidine. Each experiment was performed by concentrating the feed material to a target of 30 g/L, diafiltering against 6 diavolumes of water or 15 mM histidine, pH 5.6, and then concentrated to a target of 80 g/L. The concentrate was collected, the UF system rinsed with water (when diafiltered against water) or 15 mM hisitidine (when diafiltered against histidine), and the rinsate was added to the concentrate. The target final concentration was 60 g/L. After the experiment of diafiltration against water, the concentrated product was formulated by adding 200 mM histidine, pH 5.6 buffer at a volume ratio of 1:12.3 to meet the final 15 mM histidine concentration target.

Tables 10 and 11 summarize the attributes of PG110 BDS obtained from different UF/DF methods. The BDS pH for all four runs met the targeted range of from about 5.6 to about 6.0. The conductivities and turbidities were very similar among the four BDS samples.

TABLE 10
Comparison of PG110 BDS formulated by different methods

| | Diafiltration into water | | Diafiltration into histidine | |
|---|---|---|---|---|
| | Without | | Without | |
| | feed | With feed | feed | With feed |
| | pH | pH | pH | pH |
| Parameter | Adjustment | Adjustment | Adjustment | Adjustment |
| pH | 5.9 | 5.6 | 5.6 | 5.8 |
| Conductivity, | 1.1 | 1.1 | 1.2 | 1.0 |
| mS/cm | | | | |
| Histidine | 15.7 | 15.0 | 15.6 | 14.5 |
| Conc., mM | | | | |
| BDS Conc., | 62 | 60 | 69 | 53 |
| g/L | | | | |
| Yield, % | 91 | 94 | N.D. | 95 |

TABLE 11
Aggregates/monomer levels in PG110 feed and BDS formulated
from different processes

| | Without feed pH | | With feed pH | |
|---|---|---|---|---|
| | Adjustment | | Adjustment | |
| | Monomer | HMW | Monomer | HMW |
| Sample | % | % | % | % |
| UF/DF Feed | 96.8 | 3.2 | 96.9 | 3.1 |
| BDS (diafiltered into water), | 96.9 | 3.1 | 96.9 | 3.1 |
| at Day 0 | | | | |
| BDS (diafiltered into | 96.9 | 3.1 | 96.9 | 3.1 |
| histidine), at Day 0 | | | | |
| BDS (diafiltered into water), | 97.2 | 2.8 | 97.1 | 2.9 |
| after 2 week at 4° C. | | | | |
| BDS (diafiltered in histidine), | 97.2 | 2.8 | 96.8 | 3.2 |
| after 2 week at 4° C. | | | | |

As shown in Table 11, the SEC profiles for the BDS showed no increase in aggregates in any of the four conditions, and all samples remained very stable during an extended hold time. However, as shown in Table 12, there were significant differences in the measured turbidity profile during the diafiltration. The turbidity of retentate in experiments where the feed was adjusted to pH 5.2 was significantly (2-3 fold) lower than those not pH adjusted. For feed adjusted to pH 5.2, turbidity profiles were very comparable between the water and histidine diafiltration processes. In addition, adjusting the feed pH also appeared to reduce product losses during processing (Table 10).

TABLE 12
Normalized turbidity for PG110 during diafiltration against water and
histidine buffer

| | Normalized Retentate | | | |
|---|---|---|---|---|
| | Turbidity (NTU per g/L MAb) | | | |
| | DF into water | | DF into histidine buffer | |
| | | with | | with |
| Diavolume | without pH adj. | pH adj. | without pH adj. | pH adj. |
| 0 | 1.38 | 1.48 | 1.16 | 1.40 |
| 1 | 1.51 | 1.46 | 2.16 | 1.64 |
| 2 | 1.88 | 1.42 | 2.49 | 1.53 |
| 3 | 2.69 | 1.65 | 2.67 | 1.22 |
| 4 | 3.47 | 1.56 | N.D. | 1.34 |
| 5 | 4.27 | 1.95 | 3.22 | 1.84 |
| 6 | 5.19 | 2.05 | 3.51 | 1.57 |

Although no detectable differences in soluble aggregates were observed (Table 11), there were differences in the amount of sub-visible particles (which represents larger sizes of aggregates than those can be measured by SEC) as measured by micro-flow imaging technique (Table 13). The BDS generated from acid adjusted feed showed much lower particle counts than that from unadjusted material. Thus, reducing feed pH can effectively decrease turbidity and sub-visible particles in the retentate, which reduces the required filter area for the post UF/DF 0.2 μm filtration and potentially enhances product stability in the long term.

TABLE 13
Levels of subvisible particles in PG110 BDS obtained from different
UF/DF processes.

| Particle | Particle Counts (#/mL) | | | |
|---|---|---|---|---|
| size | DF into water | | DF into histidine buffer | |
| ranges | without pH adj. | with pH adj. | without pH adj. | with pH adj. |
| 1-2 um | 56,573 | 12,346 | 20,155 | 6,845 |
| 2-5 um | 14,929 | 3,407 | 3,642 | 854 |
| 5-10 um | 2,278 | 704 | 590 | 220 |
| 10-25 um | 350 | 205 | 145 | 40 |
| 25-100 um | 40 | 40 | 15 | 15 |

As shown in Table 14, when the feed was titrated with phosphoric acid, there was a significant increase (2-3 fold) in aggregate level as the feed pH decreases from about 7 to about 4.5. In contrast, the aggregate levels stayed constant when acetic acid or citric acid was used. Thus, the type of acid employed to adjust pH can have a dramatic impact on aggregation and turbidity formation during the UF/DF process. Acetic acid and citric acid are acids useful for PG110 feed conditioning.

When the feed pH was pre-adjusted to about 5, PG110 can be diafiltered into water without affecting the pH, conductivity, turbidity, aggregation and stability profile of the final BDS as well as the processing yield. Reducing feed pH using acids such as acetic acid and/or citric acid is an effective means to control the aggregation and particle formation during the UF/DF process for PG110 and is also likely to be effective for controlling the aggregation and particle formation during the UF/DF processes disclosed herein for other antibodies.

TABLE 14
Effect of acid types on PG110 aggregations

| Acetic Acid | | Citric Acid | | Phosphoric Acid | |
|---|---|---|---|---|---|
| pH | Aggregates % | pH | Aggregates % | pH | Aggregates % |
| 6.75 | 0.76 | 6.64 | 0.68 | 6.75 | 0.73 |
| 6.50 | 0.61 | 6.50 | 0.61 | 6.53 | 0.84 |
| 6.00 | 0.61 | 6.01 | 0.62 | 5.99 | 1.46 |
| 5.53 | 0.66 | 5.55 | 0.63 | 5.50 | 1.82 |
| 5.03 | 0.49 | 5.06 | 0.57 | 4.91 | 1.81 |
| 4.50 | 0.37 | 4.52 | 0.48 | 4.31 | 1.70 |
| 4.02 | 0.38 | 4.01 | 0.52 | 3.76 | 1.03 |

### Example 5

### 111-10

111-10 in-process feed stream obtained from a HIC polishing step (at about pH 7) was used as the load material in the UF/DF experiments described below. In a water-diafiltration experiment, the feed pH was adjusted to 5.5 using 2 M acetic acid before starting the UF/DF operation. Experiments were performed by concentrating the feed protein solution to a target of 70 g/L, diafiltering against 8 diavolumes of water or 19 mM histidine, pH 5.6, and then concentrating to a target of 195 g/L. The concentrate was collected, the UF system rinsed with water (when diafiltered against water) or 15 mM hisitidine, pH 6 (when diafiltered against histidine), and the rinsate was added to the concentrate. The target final concentration was 140±15 g/L. After the experiments of diafiltration against water, the concentrated product was formulated by adding 200 mM histidine, pH 5.8 buffer at a volume ratio of 1:12.3 to meet the final histidine concentration target.

Tables 15 and 16 showed the attributes of 111-10 BDS generated from different UF/DF processes. Overall, protein concentration and pH were all within an expected range, and there was no significant difference in the SEC and stability profiles for BDS derived from water or histidine-diafiltration processes. However, as shown in Table 17, there was a large increase in the retentate turbidity during the diafiltration into water as compared to that during diafiltration in histidine buffer for untreated feed material. Similar to PG110, once the feed pH was lowered to 5.5, the turbidity formation was mitigated, and it stayed at similar low level as that during histidine diafiltration process. Hence, when integrating feed pH adjustment 111-10 can be diafiltered into water without having an impact on final product quality and process yield.

TABLE 15
Comparison of 111-10 BDS formulated by different methods

| | Diafiltration into water | | Diafiltration into |
|---|---|---|---|
| | No feed pH | Adjust feed to | histidine (No feed |
| Parameter | adjustment | pH 5.5 | pH adjustment) |
| pH | 6.1 | 5.5 | 5.6 |
| Conductivity, mS/cm | 1.6 | 1.8 | 2.1 |
| BDS Conc., g/L | 139 | 156 | 156 |
| Yield, % | 86 | 87 | 74 |

TABLE 16
Aggregates/monomer levels in 111-10 feed and BDS formulated from
different processes

Monomer %

| Sample | @ Day 0 | After 4 week @ 4° C. |
|---|---|---|
| UF/DF Feed | 99.8 | N.D. |
| BDS (diafiltered into | 99.8 | 99.5 |
| water) | | |
| BDS (diafiltered into | 99.8 | 99.5 |
| water, adjust feed | | |
| to pH 5.5) | | |
| BDS (diafiltered into | 99.8 | 99.3 |
| histidine) | | |

TABLE 17
Normalized turbidity for 111-10 in different diafiltration processes

| | Normalized Retentate Turbidity | | | |
|---|---|---|---|---|
| | (NTU per g/L MAb) | | | |
| | DF into | | DF into | |
| | water, | | histidine, | DF into 0.01% |
| | without | DF into water, | without | Tween solution |
| Diavolume | pH adj. | with pH adj. | pH adj. | (without pH adj.) |
| 0 | 2.60 | 1.01 | 1.01 | 1.01 |
| 1 | 2.61 | 1.07 | 1.02 | 0.84 |
| 2 | 2.95 | 1.41 | 1.05 | 0.90 |
| 3 | 5.34 | 1.58 | 1.00 | N.D. |
| 4 | 5.75 | 1.44 | 1.31 | 0.91 |
| 5 | 6.38 | 1.26 | 1.09 | N.D. |
| 6 | 6.41 | 1.47 | 1.12 | N.D. |
| 7 | 7.60 | 1.47 | 1.09 | 0.88 |

The effect of pH adjustment with acids on 111-10 aggregation is depicted in Table 18. The use of acetic acid showed no impact on antibody aggregation in the pH range from 4.5 to 7. Phosphoric acid resulted in significant increase (3-5 folds) in aggregate levels in the pH range of 5-6 while citric acid had minimal effect with aggregate level increase within 0.1%. Thus, this work highlights the need for careful selection of the appropriate acid to be used for conditioning of 111-10 feed prior to UF/DF processing.

TABLE 18
Effect of acid types on 111-10 aggregations

| Acetic Acid | | Citric Acid | | Phosphoric Acid | |
|---|---|---|---|---|---|
| pH | Aggregates % | pH | Aggregates % | pH | Aggregates % |
| 7.00 | 0.10 | 7.00 | 0.13 | 7.00 | 0.10 |
| 6.52 | 0.12 | N.D. | N.D. | 6.53 | 0.17 |
| 5.82 | 0.01 | 5.47 | 0.18 | 6.00 | 0.29 |
| 5.40 | 0.09 | N.D. | N.D. | 5.49 | 0.42 |
| 5.04 | 0.11 | 5.06 | 0.19 | 5.04 | 0.44 |
| 4.53 | 0.11 | 4.50 | 0.08 | 4.36 | 0.09 |

Apart from diafiltration into water or histidine buffer, diafiltration into low level of Tween 80 aqueous solution was also evaluated for 111-10. In this case, the operating conditions were similar to those used for the other UF/DF runs described above, except for the diafiltration step. After concentrating to ˜76 g/L, the 111-10 feed was diafiltered against with 8 diavolumes of 0.01% Tween 80 solution followed by a second ultrafiltration step to reach the final protein concentration target. The retentate was spiked with a pH 5, 200 mM histidine stock buffer at volume ratio of 1:12.3. The protein solution turbidity level during the diafiltration process was measured, and the obtained BDS was analyzed for protein concentration, pH, and monomer/aggregate levels.

As shown in Table 17, the retentate turbidity stayed at a constant, low level when the protein was diafiltering into 0.01% Tween 80 solution. The final BDS has a concentration of 137 g/L at pH 6.0, with monomer level of 99.5% similar to that from the other runs. There were no operational issues such as foaming or pressure increase during this process. Thus, diafiltering into Tween solution is another viable approach to mitigate aggregation and particle formation for antibodies with a propensity for aggregation.

1. A method for preparing a formulation comprising the steps of:
a) providing a first solution, said first solution comprising one or more proteins;
b) subjecting said first solution to diafiltration, using a diafiltration solution, said diafiltration solution comprising water, until at least a five-fold volume exchange with said diafiltration solution has been achieved, thereby forming a second solution; and
c) concentrating said one or more proteins in said second solution within a range of about 10 grams per liter to about 300 grams per liter.

2. The method of claim 1, further comprising:
d) adding one or more buffer salts and/or one or more excipients to the solution of step c).

3. The method of claim 1, wherein said diafiltration solution further comprises a surfactant.

4. The method of claim 3, wherein said diafiltration solution comprises about 0.0001 percent to about 0.5 percent (w/v) polysorbate.

5. The method of claim 1, further comprising adjusting the pH of said first solution within a range of about 4 to about 6.

6. The method of claim 5, wherein said first solution further comprises acetic acid and/or citric acid in an amount sufficient to adjust the pH of said first solution within a range of about 4 to about 6.

7. The method of claim 1, wherein said concentrating step c) comprises ultrafiltering said second solution.

8. The method of claim 2, wherein said one or more buffer salts comprises histidine within a concentration range of about 10 millimolar to about 100 millimolar.

9. The method of claim 2, wherein histidine is added during any of steps a) through d) in a sufficient amount to achieve a target concentration of about 15 mM histidine at a target pH of about 6 in the formulation.

10. The method of claim 1, wherein said one or more proteins is an antibody, or antigen binding fragment thereof.

11. The method of claim 10, wherein the antibody, or antigen-binding fragment thereof, is selected from the group consisting of a chimeric antibody, a human antibody, a humanized antibody, and a domain antibody (dAb).

12. The method of claim 10, wherein the antibody, or antigen-binding fragment thereof, is an anti-TNF or an anti-IL-12 antibody.

13. The method of claim 10, wherein the antibody, or antigen-binding fragment thereof, is selected from the group consisting of Humira (adalimumab), Campath (Alemtuzumab), CEA-Scan Arcitumomab (fab fragment), Erbitux (Cetuximab), Herceptin (Trastuzumab), Myoscint (Imciromab Pentetate), ProstaScint (Capromab Pendetide), Remicade (Infliximab), ReoPro (Abciximab), Rituxan (Rituximab), Simulect (Basiliximab), Synagis (Palivizumab), Verluma (Nofetumomab), Xolair (Omalizumab), Zenapax (Daclizumab), Zevalin (Ibritumomab Tiuxetan), Orthoclone OKT3 (Muromonab-CD3), Panorex (Edrecolomab), Mylotarg (Gemtuzumab ozogamicin), golimumab (Centocor), Cimzia (Certolizumab pegol), Soliris (Eculizumab), CNTO 1275 (ustekinumab), Vectibix (panitumumab), Bexxar (tositumomab and 1131 tositumomab), Avastin (bevacizumab), 13C5.5, CPA4026, PG110, 111-10, or DVD12-1CHO.

14. The method of claim 1, wherein said one or more proteins is a therapeutic protein selected from the group consisting of Pulmozyme (Dornase alfa), Rebif, Regranex (Becaplermin), Activase (Alteplase), Aldurazyme (Laronidase), Amevive (Alefacept), Aranesp (Darbepoetin alfa), Becaplermin Concentrate, Betaseron (Interferon beta-1b), BOTOX (Botulinum Toxin Type A), Elitek (Rasburicase), Elspar (Asparaginase), Epogen (Epoetin alfa), Enbrel (Etanercept), Fabrazyme (Agalsidase beta), Infergen (Interferon alfacon-1), Intron A (Interferon alfa-2a), Kineret (Anakinra), MYOBLOC (Botulinum Toxin Type B), Neulasta (Pegfilgrastim), Neumega (Oprelvekin), Neupogen (Filgrastim), Ontak (Denileukin diftitox), PEGASYS (Peginterferon alfa-2a), Proleukin (Aldesleukin), Pulmozyme (Dornase alfa), Rebif (Interferon beta-1a), Regranex (Becaplermin), Retavase (Reteplase), Roferon-A (Interferon alfa-2), TNKase (Tenecteplase), and Xigris (Drotrecogin alfa), Arcalyst (Rilonacept), NPlate (Romiplostim), Mircera (methoxypolyethylene glycol-epoetin beta), Cinryze (C1 esterase inhibitor), Elaprase (idursulfase), Myozyme (alglucosidase alfa), Orencia (abatacept), Naglazyme (galsulfase), Kepivance (palifermin), and Actimmune (interferon gamma-1b).

15. A formulation prepared according to the method of claim 1.

16. The formulation of claim 15, wherein the formulation is stable in a liquid form for at least about 3 months.

17. The formulation of claim 15, wherein the formulation does not comprise an agent selected from the group consisting of a tonicity modifier, an anti-oxidant, a cryoprotectant, a bulking agent, and a lyoprotectant.

18. Use of the formulation of claim 15 in the treatment of a disorder in a subject comprising administering an effective amount of the formulation to a subject.

19. The use of the formulation of claim 18, wherein said disease or disorder is rheumatoid arthritis, juvenile idiopathic arthritis, psoriatic arthritis, osteoarthritis, Crohn's disease, ulcerative colitis, ankylosing spondylitis, psoriasis, multiple sclerosis, sarcoidosis, neutropenia, leukemia, lymphoma, a central neurological disorder, a peripheral neurological disorder, chronic pain, acute pain, lung cancer, stomach cancer, colon cancer, prostate cancer, brain cancer, or breast cancer.

20. A device comprising the formulation of claim 15.
